(19) United States
(12) Patent Application Publication
(10) Pub. No.: US 2007/0224447 A1
(43) Pub. Date: Sep. 27, 2007

(54) Organometallic Complex, Luminescent Solid, Organic el Element and Organic el Display

(75) Inventors: Wataru Sotoyama, Ashigarakami-gun (JP); Tasuku Satoh, Ashigarakami-gun (JP); Norio Sawatari, Kawasaki-shi (JP)

Correspondence Address: BIRCH STEWART KOLASCH & BIRCH, PO BOX 747, FALLS CHURCH, VA, 22040-0747, US

(73) Assignee: FUJIFILM Corporation, Tokyo (JP)

(21) Appl. No.: 11/579,179

(22) PCT Filed: Apr. 30, 2004
(86) PCT No.: PCT/JP04/06329

§ 371 Date: May 11, 2007

(51) Int. Cl. B32B 9/00; C07C 39/15; C07D 213/22; C07D 213/53; C07D 217/14; C07D 231/12; C07D 249/08; C07D 277/66; C07D 277/68; C07D 277/70; C07F 15/00; C07F 9/50; C09K 11/06; H05B 33/14

(52) U.S. Cl. 428/690; 546/4; 548/103; 548/402

(57) ABSTRACT

The present invention aims to provide organic EL elements etc., containing phosphorescent organometallic complexes, that can exhibit longer durability, higher emitting efficiency, superior thermal/electrical stability, significantly longer operating life. The organic EL elements comprise an organic thin layer interposed between a positive hole transport layer and a electron transport layer, the organic thin layer comprises an organometallic complex comprising at least a metal element, a tridentate ligand, and a monodentate ligand, wherein the tridentate ligand bonds with the metal atom through two nitrogen atoms and one carbon atom, and the carbon atom exists between the two nitrogen atoms, and a monodentate ligand bonds with the metal atom through a atom selected from the group consisting of C, N, O, P and S atoms.

### TECHNICAL FIELD

This application relates to organometallic complexes or luminescent solids, capable of emitting phosphorescence and appropriately utilized for organic EL elements, luminescent materials in lighting systems or color conversion materials; organic EL elements that utilize the organometallic complexes and/or luminescent solids; and EL displays that utilize the organic EL elements.

### BACKGROUND ART

Organic EL elements have typically such a construction that one or more of thin organic layer is sandwiched between a positive electrode and a negative electrode; when positive holes are injected from the positive electrode and electrons are injected from the negative electrode respectively into the organic layer, the recombination energy due to the recombination of the positive holes and the electrons causes an excitation of luminescent center of luminescent materials in the organic layer, then a light is emitted at the stage when the luminescent materials deactivate from the exciting condition to the basic condition.

The organic EL elements can exhibit characteristic features such as self-luminescence, high-speed response, excellent visibility, extra-thin model, light weight, high-speed responsibility and superior picture display, therefore, their application for flat-panel displays such as full-color displays are anticipated. After an organic EL element was reported that has a two-layer laminate construction of a positive hole-transporting organic thin film (positive hole-transport layer) and an electron-transporting organic thin film (negative hole-transport layer) in particular, such organic EL elements have been attracting much attention with respect to light emitting elements with a larger area capable of emitting at a lower voltage of 10 V or less (Non-Patent Literature 1).

A doping of a pigment molecule is proposed in order to increase the emitting efficiency of the organic EL elements; more specifically, a pigment molecule with a higher fluorescence emission is doped as a guest material into a fluorescent base material as a host material to thereby form a luminescent layer with higher emitting efficiencies (Non-Patent Literature 2).

Recently, an improvement of emitting efficiency in the organic EL elements are reported, in which a phosphorescent material that can emit from the molecular excited-triplet state is utilized as the luminescent material of the organic EL elements in place of the previous phosphorescent materials, the improvement has been attracting attention (Non-Patent Literature 3, Non-Patent Literature 4). Light emissions from organic materials are classified into fluorescence and phosphorescence depending of the excited states that cause the emission. Previously, fluorescent materials have been employed in the organic EL elements by reason that conventional organic materials emit no phosphorescence at room temperature. In view of EL emission mechanism, it is estimated that the phosphorescent state occurs in four times higher probability of the fluorescent state, thus there recently exists much interest in the application of heavy metal complexes capable of emitting phosphorescence at room temperature in order to enhance the emitting efficiency of EL elements. However, phosphorescent materials suffer from poor margin in nominating the materials since there exist few materials that emit strong phosphoresce at room temperature.

An example of publicly known organometallic complexes, utilized for organic EL elements phosphorescent at room temperature, is the metal complex having a (N,N,C)-tridentate ligand containing two coordinate bonds of Pt and N atoms and one direct coupling of Pt and C atoms (Patent Literature 1). However, the metal complex exhibits an insufficient emitting efficiency and thus the organic EL elements with the metal complex are likely to represent lower emitting efficiencies. On the other hand, it is reported that a Pt complex containing a (N,C,N)-tridentate ligand and a Cl atom can emit phosphorescence in a solution at a higher efficiency than the Pt complex with the (N,N,C)-tridentate ligand (Non-Patent Literature 5). However, the organic EL elements with the metal complex suffer from shorter operating life.
Non-Patent Literature 1: C. W. Tang and S. A. VanSlyke, Applied Physics Letters vol. 51, 913 (1987)
Non-Patent Literature 2: C. W. Tang, S. A. VanSlyke, and C. H. Chen, Journal of Applied Physics vol. 65, 3610 (1989)
Non-Patent Literature 3: M. A. Baldo, et al., Nature vol. 395, 151 (1998)
Non-Patent Literature 4: M. A. Baldo, et al., Applied Physics Letters vol. 75, 4, (1999)
Non-Patent Literature 5: J. A. G. Williams et al., Inorganic Chemistry, Vol. 42, 8609-8611 (2003)
Patent Literature 1: Japanese Patent Application Laid-Open UP-A) No. 2002-363552

It is an object of the present invention to provide an organometallic complex capable of emitting phosphorescence and appropriately utilized for organic EL elements, luminescent materials in lighting systems, color conversion materials etc.; it is another object of the present invention to provide a luminescent solid that contains the organometallic complex; it is still another object of the present invention to provide an organic EL element, containing the organometallic complex and/or the luminescent solid, that can exhibit longer durability, higher emitting efficiency, superior thermal/electrical stability, significantly longer operating life; it is still another object of the present invention to provide an organic EL display, containing the organic EL element, that can exhibit higher performance and longer durability, represent a constant average driving current regardless of the luminous pixel, be appropriately utilized for full-color displays with excellent color balance without changing the emitting area, and represent longer operating life.

### DISCLOSURE OF INVENTION

The present inventors have investigated vigorously to solve the problems described above and have found as follows: a metal complex, containing a metal atom, a (N,C,N)-tridentate ligand and a specific monodentate ligand, can emit strong phosphorescence, provide the organic EL element with a proper sublimating property, and make possible to vapor-deposit neat films or dope films, and are suitable for luminescent materials in organic EL elements or lighting systems; and the organic EL element and the organic EL display, which utilize the organometallic complex, are excellent in terms of longer durability, higher emitting efficiency, superior thermal/electrical stability, and significantly longer operating life. The present invention is based on the discoveries described above; the means for solving the problems will be explained in the following.

The organometallic complex according to the present invention is characterized in that it comprises a metal atom, a tridentate ligand that bonds with the metal atom through two nitrogen atoms and one carbon atom in which the carbon atom exists between the two nitrogen atoms, and a monodentate ligand that bonds with the metal atom through a atom selected from the group consisting of C, N, O, Si, P and S atoms.

Light emissions from organic materials are classified into fluorescence and phosphorescence depending of the excited states that cause the emission. Previously, fluorescent materials have been employed in the organic EL elements, luminescent materials of lighting systems, and color conversion materials by reason that conventional organic materials typically emit no phosphorescence at room temperature. In view of EL emission mechanism, on the contrary, it is estimated that the phosphorescent state occurs in four times higher probability of the fluorescent state, thus there recently exists much interest in the application of metal complexes capable of emitting phosphorescence at room temperature in order to enhance the emitting efficiency of EL elements. The organometallic complex according to the present invention can emit strong phosphorescence, therefore, an emitting efficiency up to 100% can be achieved theoretically while internal quantum efficiency of EL elements of fluorescent materials is 25% at most. Accordingly, the organometallic complexes capable of emitting strong phosphorescence can be appropriately utilized for the emitting materials of organic EL elements etc. The organometallic complexes according to the present invention can change its emitting color by changing the skeleton structure, species or number of substituents etc. of the specific (N,C,N)-tridentate ligand and the monodentate ligand.

The inventive luminescent solids contain the inventive organometallic complexes. The inventive luminescent solids, which containing the inventive organometallic complexes, can exhibit significantly longer operating life, superior durability and high efficiency, thus can be appropriately utilized for lighting systems, display systems etc.

The inventive organic EL elements are equipped with an organic thin layer between a positive electrode and a negative electrode, and the organic thin layer contains the organometallic complex. The inventive organic EL elements can exhibit significantly longer operating life, superior durability and high efficiency, thus can be appropriately utilized for lighting systems, display systems etc.

The inventive organic EL displays utilize the inventive organic elements. The inventive organic EL displays can exhibit significantly longer operating life, superior durability and high efficiency.

### BRIEF DESCRIPTION OF DRAWINGS

FIG. 1 is a schematic view that explains exemplarily a layer construction in an organic EL element according to the present invention.

FIG. 2 is a schematic view that explains exemplarily a construction of an organic EL display.

FIG. 3 is a schematic view that explains exemplarily a construction of an organic EL display.

FIG. 4 is a schematic view that explains exemplarily a construction of an organic EL display.

FIG. 5 is a schematic view that explains exemplarily a construction of an organic EL display of passive matrix system (passive matrix panel).

FIG. 6 is a schematic view that explains exemplarily a circuit in the organic EL display of passive matrix system (passive matrix panel) shown in FIG. 5.

FIG. 7 is a schematic view that explains exemplarily a construction of an organic EL display of active matrix system (active matrix panel).

FIG. 8 is a schematic view that explains exemplarily a circuit in the organic EL display of active matrix system (active matrix panel) shown in FIG. 7.

FIG. 9 is an IR spectrum of Pt(3,5-di(2-pyridyl)toluene)(biphenyloxide).

FIG. 10 is an IR spectrum of Pt(3,5-di(2-pyridyl)toluene)(OH).

FIG. 11 is an IR spectrum of Pt(3,5-di(2-pyridyl)toluene)(1,2,4-triazolate).

FIG. 12 is an IR spectrum of Pt(3,5-di(2-pyridyl)toluene)(2-benzothiazolate).

FIG. 13 is an IR spectrum of Pt(3,5-di(2-pyridyl)toluene)(phenylacetylide).

FIG. 14 is a schematic view that explains an outline for determining a phosphorescence quantum yield.

FIG. 15 is an ER spectrum of an organic EL element of Example 14.

### BEST MODE FOR CARRYING OUT THE INVENTION

Organometallic Complex and Luminescent Solid

The inventive organometallic complex comprises a metal atom, a tridentate ligand that bonds with the metal atom at three sites, and a monodentate ligand that bonds with the metal atom at one site.

The luminescent solid of the present invention comprises the organometallic complex of the present invention and other components as required. The condition of the luminescent solid may be properly selected depending on the purpose; for example, the luminescent solid may be a crystal, a thin film or the like. The content of the organometallic complex in the luminescent solid may be properly selected depending on the purpose; usually, the content is 0.1% by mass to 50% by mass, preferably 0.5% by mass to 20% by mass in order to obtain higher emitting efficiency.

Metal Atom

The metal atom acts as a center metal in the organometallic complex. The metal atom may be properly selected depending on the purpose; examples thereof include Fe, Co, Ni, Ru, Rh, Pd, Os, Ir, Pt etc. Each of these metal atoms exists as one atom per molecule of the organometallic complex. One or plural species of metal atoms may exist in the plural molecules of the organometallic complexes. It is preferred in particular that the metal atom is Pt among these metal atoms and the organometallic complex is a Pt complex.

Tridentate Ligand

The tridentate ligand may be properly selected without limitation from (N,C,N)-tridentate ligands capable of bonding with the metal atom at three sites through two nitrogen atoms and one carbon atom, and the carbon atom exists between the two nitrogen atoms (hereinafter, sometimes distinguished by “first nitrogen atom” and “second nitrogen atom”). It is preferred that the three atoms of the two nitrogen atoms and the one carbon atom in the tridentate ligand are each a part of ring structures which are different each other. More preferably, the first nitrogen-adjacent atom (i.e. atom adjacent to the first nitrogen atom in the ring structure) bonds to the first carbon-adjoining atom (i.e. the first atom adjoining to the carbon atom in the ring structure), and the second nitrogen-adjacent atom (i.e. atom adjacent to the second nitrogen atom in the ring structure) bonds to the second carbon-adjoining atom (i.e. the second atom adjoining to the carbon atom in the ring structure). It is particularly preferable that the first carbon-adjoining atom and the second carbon-adjoining atom are each a carbon atom.

Monodentate Ligand

The monodentate ligand may be properly selected without limitation from those capable of bonding with the metal atom through an atom selected from the group consisting of C, N, O Si, P and S atoms. Preferably, the electrical charge of the organometallic complex can be neutralized by the monodentate ligand so as to provide the organometallic complex with subliming ability.

Specific Example of Organometallic Complex

Specific examples of the organometallic complexes in the present invention are those expressed by the general formula (1) below.

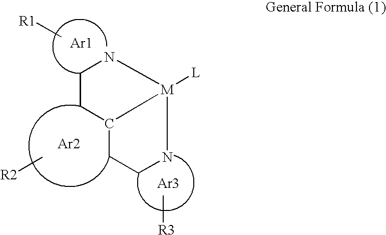

in the general formula (1), M represents one selected from the metal atoms described above; Ar1, Ar2 and Ar3 are each a ring structure, which is selected from five-membered ring groups, six-membered ring groups and condensed ring groups thereof. Preferably, Ar2 is one of benzene ring structures, pyridine ring structures, pyrimidine ring structures and pyrene ring structures. Specifically, the following structures are more preferable.

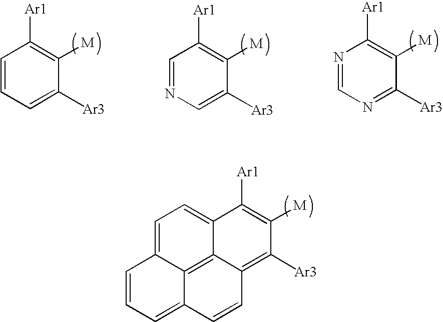

in the above formulas, M represents one selected from the metal atoms described above, Ar1 and Ar2 are each a ring structure selected from those described above.

Preferably, one of Ar1 and Ar3 is one of monocyclic heteroaromatic groups and polycyclic heteroaromatic groups, specific examples are those indicated below. Ar1 and Ar3 may be identical or different each other, preferably identical.

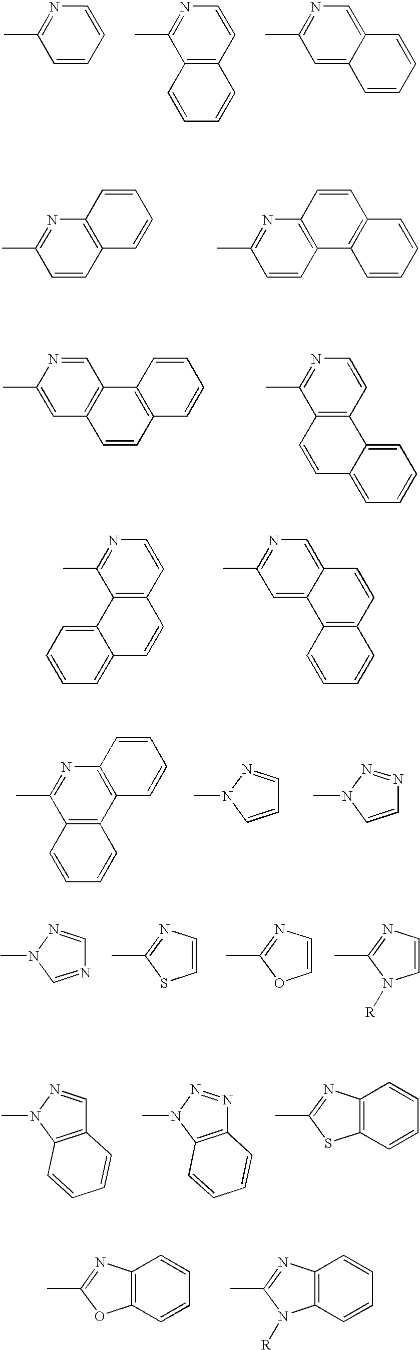

R1, R2 and R3 represent each a substituent or hydrogen atom that substitutes Ar1, Ar2 and Ar3 respectively. R1, R2 and R3 may be identical or different, singular or plural, or neighbors thereof may bond to form a ring. Specific examples of R1, R2 and R3 are a halogen atoms, cyano group, alkoxy group, amino group, alkyl group, alkyl acetate group, cycloalkyl group, aryl group, aryloxy group and the like, which may be further substituted by other substituents.

L represents a monodentate ligand that bonds to the metal atom M through an atom selected from C, N, O P and S atoms.

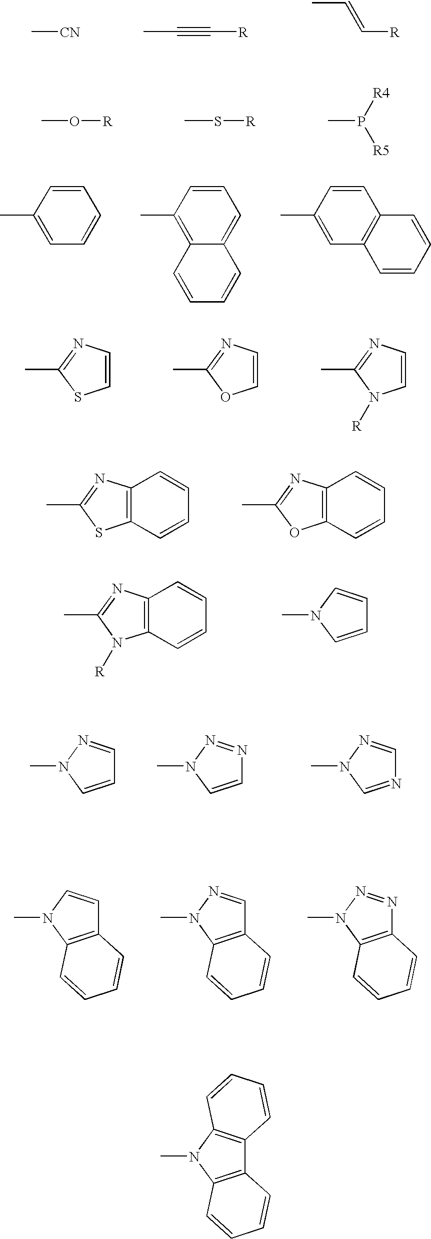

In these groups, hydrogen atoms may be substituted by an organic group or halogen atom; R represents a hydrogen atom, alkyl group or aryl group; R4 and R5 represent each one of hydrogen atom, alkyl group, aryl group, alkoxy group and aryloxy group.

The organometallic complexes expressed by the general formula (1) described above are electrically neutral and can sublime under vacuum, therefore, can be advantageously formed into a thin film by a vacuum vapor-deposition process in addition to conventional coating processes.

The organometallic complexes expressed by the general formula (1), in which Ar2 is a benzene ring structure, are as follows:

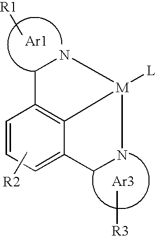

The structure of the organometallic complexes, in which both of the Ar1 and Ar3 being a benzene ring structure, is as follows:

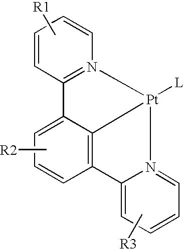

The relative quantum yield of photoluminescence (sometimes referred to as “PL”) for organometallic complexes of the present invention is preferably no less than 70% measured in a film form, more preferably no less than 90%, based on that of the aluminumquinoline complex (Alq₃) thin film (PL quantum yield=22%) of the same thickness.

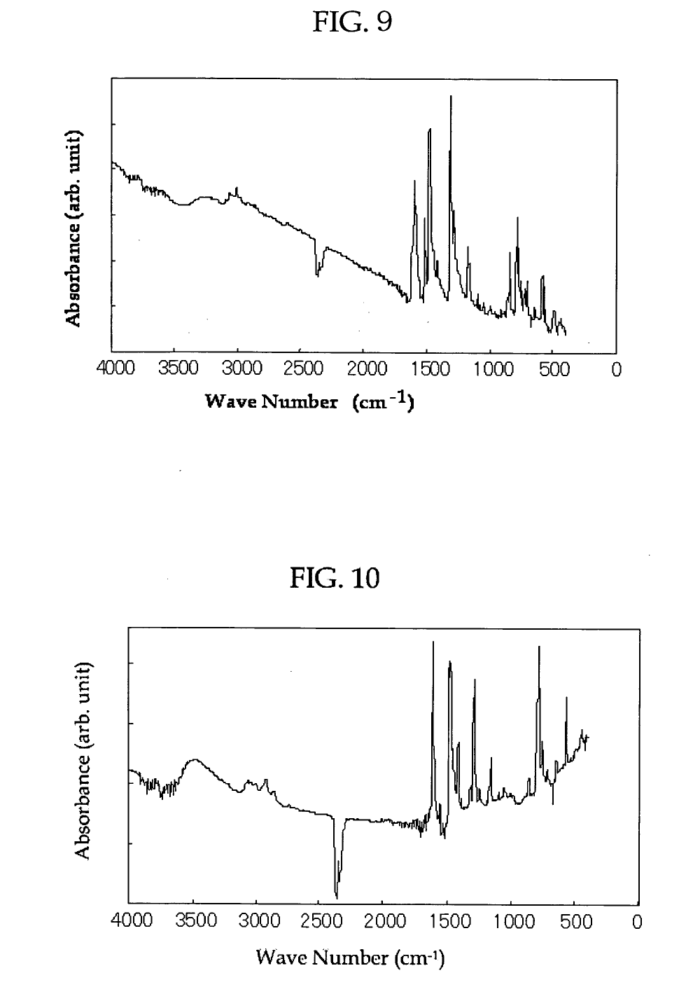

The PL quantum yield, for example, can be determined as follows. That is, an excitation light 100 (constant light of 365 nm) from a light source is illuminated slantingly on thin film 102 on a transparent substrate as shown in FIG. 9, and the PL photon number [P(sample)] is calculated by conversing the PL spectrum of the thin film measured by spectroradiometer 104 (Konica Minolta, CS-1000). At the same time with the measurement of the light emission, the total intensity [I(sample)] of the light, reflected collectively by mirror 106 which being transmitted and reflected from the sample, is detected by the photodiode 108. Subsequently, the same measurement was also carried out on the Alq₃ thin film (PL quantum yield=22%) as a reference to determine the PL photon number [P(ref.)] and total intensity [I(ref.)] of the reflected and transmitted lights. Then the total intensity [I(substrate)] of the reflected and transmitted lights is determined for a transparent substrate itself. The PL quantum yield of thin film sample can be calculated from the following formula.

$$( P L quantum efficiency ) = \frac{P ( sample ) / [ I ( substrate ) - I ( sample ) ]}{P ( ref . ) / [ I ( substrate ) - I ( ref . ) ]} \times 22 \%$$

The synthesis method of organometallic complexes according to the present invention may be properly selected depending on the purpose; for example, an organometallic complex as a precursor having the (N,C,N)-tridentate ligand, the metal atom and a halogen atom such as a chlorine atom is reacted with a halogen-substituted or alkali metal-substituted monodentate ligand described above in accordance with conditions selected suitably.

The above reaction may be achieved by employing a catalyst; the catalyst may be properly selected depending on the purpose, suitable examples of the catalyst are copper salt-organic amines. These may be utilized alone or in combination.

The synthesis method for the organometallic complex having the tridentate ligand, the metal atom and the halogen atom such as chlorine atom may be properly selected depending on the purpose; suitable processes are exemplified in “D. J. Cardenas and A. M. Echavarren, Organometallics Vol. 18, 3337 (1999)” and the like.

The organometallic complexes of the present invention have excellent PL quantum yields and exhibit higher luminous efficiency as mentioned above, thus can be appropriately utilized in various fields; among others, they can be utilized for organic EL elements and lighting systems from the viewpoint of higher luminance brightness and longer lifetime. Furthermore, the organic EL displays containing the organic EL elements typically involve the combination of red, green and blue organic EL elements as one pixel in order to produce full-color displays, therefore, there needs organic EL elements for three colors. The organometallic complexes according to the present invention can be advantageously applied to the organic EL elements from the viewpoint that organometallic complexes can change or adjust their colors by changing the molecular structure of the tridentate ligands to emit the respective colors of red, green and blue.

Organic EL Element

The organic EL elements according to the present invention comprise an organic thin film layer interposed between a positive electrode and a negative electrode, and the organic thin film layer contains the organometallic complex according to the present invention and also other layers or materials as required.

The organic thin film layer may be properly selected depending on the purpose; for example, the organic thin film layer comprises at least the light emitting layer and may comprise light emitting layer, a positive hole injection layer, a positive hole transport layer, a positive hole blocking layer, an electron transport layer, or an electron injection layer as required. The light emitting layer may be prepared as a single function for the light emitting layer or as multiple functions, for example, for light emitting layer/electron transport layer or light emitting layer/positive hole transport layer.

Light Emitting Layer

The light emitting layer may be properly selected depending on the purpose; preferably, the light emitting layer contains the organometallic complexes according to the present invention as a luminescent material. The light emitting layer may be formed into a film from the organometallic complexes themselves, alternatively, a combination of the inventive organometallic complex as a guest material and another material as a host material may be formed into a film provided that the host material has an emission wavelength around the absorbing wavelength of the guest material. It is preferred that the host material is contained in the light emitting layer, alternatively the host material may be contained in the positive hole transport layer or the electron transport layer.

In cases where the combination of the inventive organometallic complex as a guest material and another material as a host material is employed, the host material is initially excited and the EL light is emitted. Since there exist a common region between the emission wavelength of the host material and the absorbing wavelength of the guest material of the organometallic complexes, the exciting energy efficiently transfers from the host material to the guest material, the host material returns to its ground state without emitting light, and only the excited guest material emits the excitation energy as the light. Therefore, this material excels in luminous efficiency, color purity and the like.

In cases where light emitting molecules exist in thin films solely or at higher contents, the decrease of emitting efficiency so-called “concentration quenching” is likely to take place due to the interaction between the light emitting molecules. When the combination of the guest material and the host material is employed, the organometallic complex of the guest material is diluted into a relatively lower concentration in the host material, therefore, it is beneficial that the “concentration quenching” can be effectively suppressed and the emitting efficiency can be higher. Furthermore, when the combination of the guest material and the host material is employed in the light emitting layer, it is beneficial that the film can be easily produced while maintaining the emitting properties since the host material typically provides higher processability in the film production.

The host material may be properly selected depending on the purpose; preferably, the emission wavelength of the host material is around the emission wavelength of the guest material. Examples of the host material include aromatic amine derivatives expressed by the structural formula (1) below, carbazole derivatives expressed by the structural formula (2) below, oxine complexes expressed by the structural formula (3) below, 1,3,6,8-tetraphenylpyrene compounds expressed by the structural formula (4) below, 4,4′-bis(2,2′-diphenylvinyl)-1,1′-biphenyl (DPVBi) (main emission wavelength=470 nm) expressed by the structural formula (5) below, p-sexiphenyl expressed by the structural formula (6) below, 9,9′-bianthryl (main emission wavelength=400 nm) expressed by the structural formula (7) below, and polymer materials mentioned later.

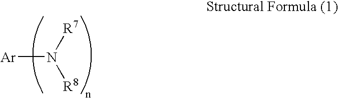

In the structural formula (1) described above, n represents an integer of 2 or 3; Ar represents a divalent or a trivalent aromatic group or heterocyclic aromatic group; R⁷ and R⁸ may be identical or different and represent a monovalent aromatic group or a heterocyclic aromatic group. The aforementioned monovalent aromatic group or heterocyclic aromatic group may be properly selected depending on the purpose.

Among the aromatic amine derivatives represented in the aforementioned structural formula (1), preferable are N,N′-dinaphthyl-N,N′-diphenyl-[1,1′-biphenyl]-4,4′-diamine (NPD) (main emission wavelength=430 nm) and its derivatives expressed by the structural formula (1)-1 below.

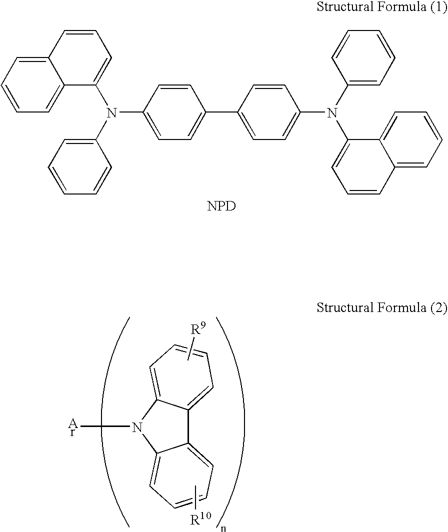

In the structural formula (2), Ar represents a divalent or a trivalent group containing an aromatic ring, or a divalent or a trivalent group containing a heterocyclic aromatic ring.

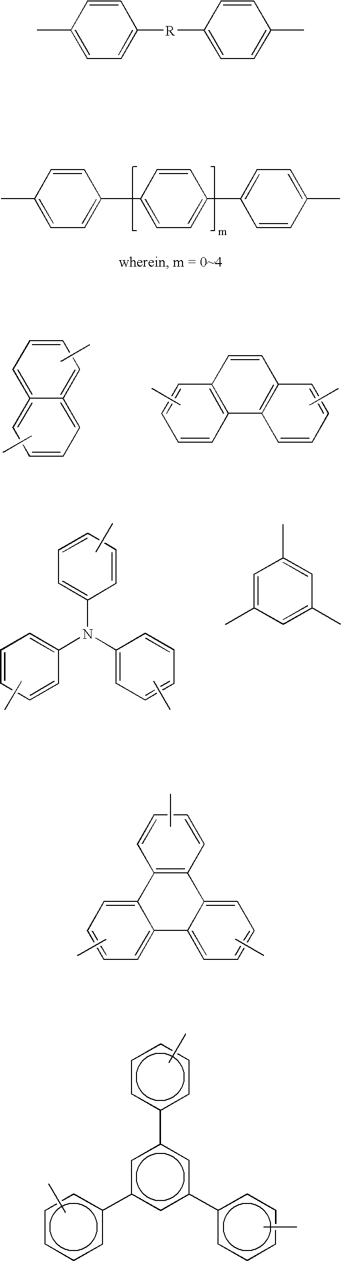

These may be substituted by non-conjugated groups. R represents a linking group, for example, the following groups are suitable.

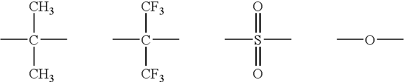

In the structural formula (2) described above, R⁹ and R¹⁰ represent each independently a hydrogen atom, halogen atom, alkyl group, aralkyl group, alkenyl group, aryl group, cyano group, amino group, acyl group, alkoxycarbonyl group, carboxyl group, alkoxy group, alkylsulfonyl group, hydroxyl group, amide group, aryloxy group, aromatic hydrocarbon or aromatic heterocyclic group; and these may be further substituted by a substituent group.

In the structural formula (2) described above, n represents an integer, preferably n is 2 or 3. Among the carbazole derivatives represented by the structural formula (2), preferable are those in which Ar is an aromatic group of which two benzene rings are joined via a single bond, R⁹ and R¹⁰ are each a hydrogen atom, and n=2; that is, preferable are 4,4′-bis(9-carbazolyl)-biphenyl (CBP) (main emission wavelength=380 nm) and its derivatives, expressed by the following structural formula (2)-1, due to higher emitting efficiency.

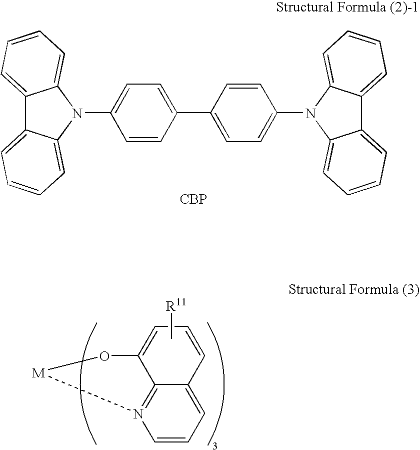

In the structural formula (3) described above, R¹¹ represents a hydrogen atom, halogen atom, alkyl group, aralkyl group, alkenyl group, aryl group, cyano group, amino group, acyl group, alkoxycarbonyl group, carboxyl group, alkoxy group, alkylsulfonyl group, hydroxyl group, amide group, aryloxy group, aromatic hydrocarbon or aromatic heterocyclic group. These may be further substituted by a substituent.

Among the oxine complexes expressed by the structural formula (3), the aluminum quinoline complex (Alq) (main emission wavelength=530 nm) represented by the following structural formula (3)-1 is preferable.

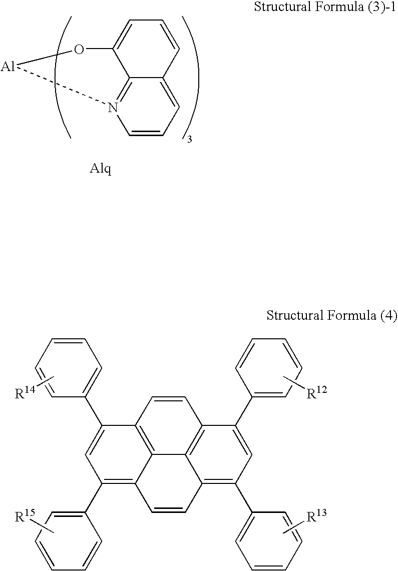

In the structural formula (4), R¹² to R¹⁵, which may be identical or different each other, represent each a hydrogen atom or a substituent. Examples of the substituent group are alkyl groups, cycloalkyl groups or aryl groups, which may be further replaced by substituents.

Among the 1,3,6,8-tetraphenylpyrene compounds expressed by the structural formula (4), the compounds of which R¹² to R¹⁵ are each hydrogen atom, that is, 1,3,6,8-tetraphenylpyrene (main emission wavelength=440 nm) expressed by the structural formula (4)-1 is preferable due to higher emitting efficiency.

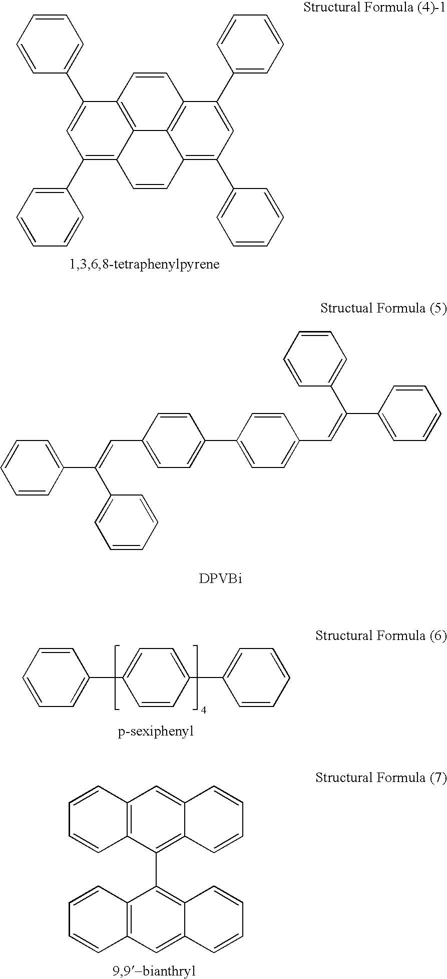

The host material of the polymer material may be properly selected depending on the purpose; preferably, the host material is selected from poly(p-phenylenevinylene) (PPV), polythiophene (PAT), poly(p-phenylene) (PPP), poly(vinyl carbazole) (PVCz), polyfluorene (PF), polyacetylene (PA) and their derivatives.

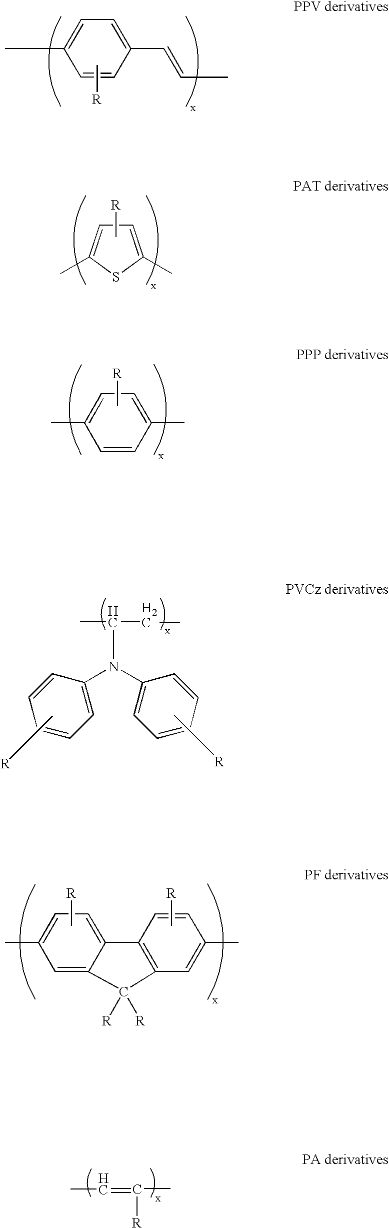

In the structural formulas described above, R represents a hydrogen atom, halogen atom, alkoxy group, amino group, alkyl group, cycloalkyl group, aryl group having optionally a nitrogen atom or sulfur atom, or an aryloxy group, which may be substituted by a substituent group; x represents an integer.

Among the host materials of polymer materials, poly(vinylcarbazole) (PVCz), expressed by the following structural formula (8), is preferable from the viewpoint that the energy transfer from the host material to the guest material proceeds efficiently.

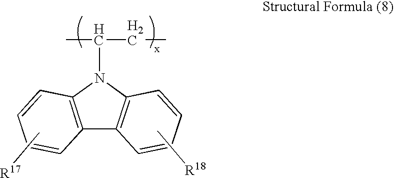

Each of R¹⁷ and R¹⁸ in the structural formula (8) is plural substituents at optional positions of the ring structure, and represents independently a hydrogen atom, halogen atom, alkoxy group, amino group, alkyl group, cycloalkyl group, aryl group having optionally a nitrogen atom or sulfur atom, or an aryloxy group, which may be substituted by a substituent group. The optional adjoining substituents of R¹⁷ and R¹⁸ may bond to form an aromatic ring that may contain a nitrogen, sulfur or oxygen atom, which may be substituted by a substituent group; x represents an integer.

In cases where the host material of the polymer material is employed, the host material is dissolved in a solvent, to which the guest material of the inventive organometallic complex is mixed to prepare a coating liquid, which is then coated by wet film-forming processes such as spin-coat processes, ink-jet processes, dip-coat processes and blade-coat processes. For the purpose of enhancing the charge-transporting property of the resulting layer, a material for the positive hole transport layer and a material for the electron transport layer may be compounded with the solution thereby to form a film. These wet film-forming processes may be preferably employed to form a multi-functional light emitting layer such as positive hole transport layer/electron transport layer/light emitting layer into one layer.

The content of the organometallic complex in the light emitting layer may be properly selected depending on the purpose; preferably, the content is 0.1% by mass to 50% by mass, and more preferably 0.5% by mass to 20% by mass. In cases where the content is less than 0.1% by mass, the lifetime and the emitting efficiency may be insufficient, and when the content is more than 50% by mass, the color purity may deteriorate. On the other hand, the content within the above preferred range may bring about advantages in lifetime, emitting efficiency etc.

The preferable content of the organometallic complex may be substantially the same in cases where the light emitting layer is formed into multi-functional such as positive hole transport layer/electron transport layer/light emitting layer into one layer.

Upon applying an electric field, positive holes can be injected from the positive electrode, positive hole-injection layer, positive hole-transport layer etc. into the light emitting layer, electrons can be injected from the negative electrode, electron injection layer, electron transport layer etc. into the light emitting layer. In addition, the light emitting layer performs to allow the recombination between the positive holes and the electrons and to emit a light from the organometallic complex as the emitting material or luminescent molecule by use of the recombination energy. The light emitting layer may contain the other emitting materials in addition to the organometallic complex within an appropriate range harmless to the emission.

The light emitting layer can be formed in accordance with conventional processes such as vapor deposition processes, wet film-forming processes, molecular beam epitaxy processes, cluster ion beam processes, molecule laminating processes, LB processes, printing processes, transfer processes, and the like.

Among these, vapor deposition processes are preferable from the viewpoint that no organic solvent is used thus no waste liquid generates and the production is relatively of lower cost, simple and efficient. In cases where the light emitting layer is formed into a single layer structure such as positive hole transport layer/light emitting layer/electron transport layer, the wet film forming processes may be available.

The vapor deposition processes may be properly selected depending on the purpose; more specifically, the processes may be vacuum vapor deposition processes, resistance heating vapor deposition processes, chemical vapor deposition processes, physical vapor deposition processes and the like. Examples of chemical vapor deposition are plasma CVD, laser CVD, heat CVD and gas source CVD. The light emitting layer may be formed by the vapor deposition processes, for example, by vapor-depositing the organometallic complexes. In cases where the light emitting layer contains the host material in addition to the organometallic complex, the light emitting layer may be advantageously formed by vacuum vapor-depositing the organometallic complex and the host material simultaneously. The former process described above is relatively easy since the simultaneous vapor deposition is unnecessary.

The wet film forming processes may be properly selected from conventional ones; examples thereof include ink-jet processes, spin coating processes, kneader coating processes, bar coating processes, braid coating processes, casting processes, dipping processes, curtain coating processes and the like.

In the wet film forming processes, a solution that dissolves or disperses the material of the light emitting layer and a resin may be utilized or coated. Examples of the resin include polyvinyl carbazole, polycarbonate, polyvinyl chloride, polystyrene, polymethyl methacrylate, polyester, polysulfone, polyphenylene oxide, polybutadiene, hydrocarbon resins, ketone resins, phenoxy resins, polyamide, ethyl cellulose, vinyl acetate, ABS resins, polyurethane, melamine resins, unsaturated polyester resins, alkyde resins, epoxy resins, silicone resins and the like.

In the wet film forming processes, the light emitting layer may be formed by preparing a solution from the organometallic complex, optional resin, and a solvent then coating and drying the solution. In cases where the light emitting layer contains the host material in addition to the organometallic complex, the light emitting layer may be formed by preparing a solution from the organometallic complex, host material, optional resin, and a solvent then coating and drying the solution.

The thickness of the light emitting layer may be properly selected depending on the purpose; preferably, the thickness is 1 nm to 50 nm, more preferably 3 nm to 20 nm.

In cases where the thickness of the light emitting layer is within the above preferable range, the emitting efficiency, luminance brightness, and color purity emitted from the organic EL element may be satisfied, and in cases within the more preferable range, these effects are more significant.

Positive Electrode

The positive electrode may be properly selected depending on the purpose; it is preferred that the positive electrode can supply the positive holes or carriers into the organic thin layer, more specifically, into the light emitting layer in cases where the organic thin layer contains only the light emitting layer, into the positive hole transport layer in cases where the organic thin layer contains further the positive hole transport layer, into the positive hole injection layer in cases where the organic thin layer contains still further the positive hole injection layer.

The material of the positive electrode may be properly selected depending on the purpose; examples thereof include metals, alloys, metal oxides, electrically conductive compounds, mixtures thereof and the like; among these, materials having a work function of 4 eV or more are preferred.

Specific examples of the material of the positive electrode are electrically conductive metal oxides such as tin oxide, zinc oxide, indium oxide and indium tin oxide (ITO); metals such as gold, silver, chromium and nickel; mixtures or laminates of these metals and electrically conductive metal oxides; inorganic electrically conductive substances such as copper iodide and copper sulfide; organic electrically conductive materials such as polyaniline, polythiophene and polypyrrole; laminates of these with ITO, and the like. These may be used alone or in combination. Among these, electrically conductive metal oxides are preferred, and ITO is particularly preferred from the viewpoint of higher productivity, higher conductivity and transparency.

The thickness of the positive electrode may be selected depending on the material; preferably, the thickness is 1 nm to 5000 nm, more preferably 20 nm to 200 nm. The positive electrode is typically formed on a substrate such as glasses like soda lime glass or non-alkali glass or transparent resins.

When using the glass as the substrate, non-alkali glass or soda lime glass with a barrier coat of silica etc. is preferred from the viewpoint of less eluting ions from the glass.

The thickness of the substrate may be properly selected to provide a sufficient mechanical strength; when using glasses as the substrate, the thickness is usually 0.2 mm or more, preferably 0.7 mm or more.

The positive electrode can be properly produced by applying a ITO substance in accordance with the processes described above such as vapor deposition processes, wet film forming processes, electron beam processes, sputtering processes, reactant sputtering processes, MBE (molecular beam epitaxy) processes, cluster ion beam processes, ion plating processes, plasma polymerization processes (high frequency excitation ion plating processes), molecule laminating processes, LB processes, printing processes, transfer processes and chemical reaction processes (e.g. sol gel process).

The drive voltage of the organic EL elements may be reduced or the emitting efficiency may be increased by way of washing or other treating the positive electrode. Examples of the other treatment are appropriately exemplified by UV ozonization and plasma processing in cases where the material of the positive electrode is ITO.

Negative Electrode

The negative electrode may be properly selected depending on the purpose; it is preferred that the negative electrode can supply the electrons into the organic thin layer, more specifically, into the light emitting layer in cases where the organic thin layer contains only the light emitting layer, into the electron transport layer in cases where the organic thin layer contains further the electron transport layer, into the electron injection layer in cases where the organic thin layer contains still further the electron injection layer.

The material of the negative electrode may be properly selected depending on the adhesion properties with adjacent layers or molecules such as the electron transport layer and light emitting layer, ionization potential, stability etc. Examples of the material include metals, alloys, metal oxides, electrically conductive compounds, mixtures thereof and the like.

Examples of the material of the negative electrode are alkali metals such as Li, Na, K and Cs; alkaline earth metals such as Mg and Ca; gold, silver, lead, aluminum, sodium-potassium alloys or mixtures thereof, lithium-aluminum alloys or mixtures thereof, magnesium-silver alloys or mixtures thereof, rare earth metals such as indium and ytterbium, and their alloys and the like.

These may be used alone or in combination. Among these, materials having a work function of 4 eV or less are preferred; more preferable are aluminum, lithium-aluminum alloys or mixtures thereof, magnesium-silver alloys or mixtures thereof etc.

The thickness of the negative electrode may be properly selected depending on the material thereof etc.; preferably, the thickness is 1 nm to 10000 nm, more preferably 20 nm to 200 nm.

The negative electrode can be properly produced by vapor deposition processes, wet film forming processes, electron beam processes, sputtering processes, reactant sputtering processes, MBE (molecular beam epitaxy) processes, cluster ion beam processes, ion plating processes, plasma polymerization processes (high frequency excitation ion plating processes), molecule laminating processes, LB processes, printing processes and transfer processes

In cases where two or more materials are employed for the negative electrode, the two or more materials may be vapor-deposited together with to form an alloy electrode, or a prepared alloy may be vapor-deposited to form an alloy electrode. The resistance of the positive electrode and the negative electrode is preferred to be lower such as no more than a few hundred ohms/square.

Positive Hole Injection Layer

The positive hole injection layer may be properly selected depending on the application, preferably, from those capable of injecting positive holes from the positive electrode upon applying an electric field.

The material of the positive hole injection layer may be properly selected depending on the purpose; preferable examples thereof include a starburst amine (4,4′,4″-tris(2-naphthylphenylamino)triphenylamine) (hereinafter sometimes referred to as “2-TNATA”) expressed by the following formula, copper phthalocyanine, polyaniline etc.

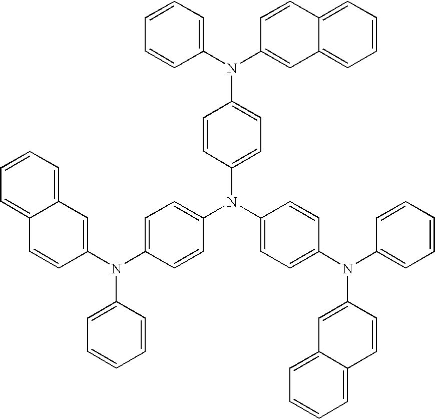

The thickness of the positive hole injection layer may be properly selected depending on the application; preferably, the thickness is 1 nm to 100 nm, more preferably 5 nm to 50 nm.

The positive hole injection layer may be properly formed by, for example, vapor deposition processes, wet film forming processes, electron beam processes, sputtering processes, reactant sputtering processes, MBE (molecular beam epitaxy) processes, cluster ion beam processes, ion plating processes, plasma polymerization processes (high frequency excitation ion plating processes), molecule laminating processes, LB processes, printing processes and transfer processes.

Positive Hole Transport Layer

The positive hole transport layer may be properly selected depending on the application, preferably, from those capable of transporting positive holes from the positive electrode upon applying an electric field.

The material of the positive hole transport layer may be properly selected depending on the purpose; examples thereof include aromatic amine compounds, carbazole, imidazole, triazole, oxazole, oxadiazole, polyarylalkane, pyrrazoline, pyrrazolone, phenylene diamine, arylamine, amine-substituted calcone, stylyl anthracene, fluorenone, hydrazone, stylbene, silazane, stylyl amine, aromatic dimethylidene compounds, porphyrine compounds, polisilane compounds, poly(N-vinylcarbazole), aniline copolymers, electrically conductive oligomers and polymers such as thiophene oligomers and polymers, polythiophene and carbon film. When the material of the positive hole transport layer is combined with the material of the light emitting then to form a positive hole transport layer, the resulting layer may also perform as a light emitting layer.

These may be used alone or in combination of two or more. Among these, aromatic amine compounds are preferred; more specifically, TPD (N,N′-diphenyl-N,N′-bis(3-methylphenyl)-[1,1′-biphenyl]-4,4′-diamine) expressed by the structural formula below, and NPD (N,N′-dinaphthyl-N,N′-diphenyl-[1,1′-biphenyl]-4,4′-diamine) expressed by the structural formula (67) below are preferable.

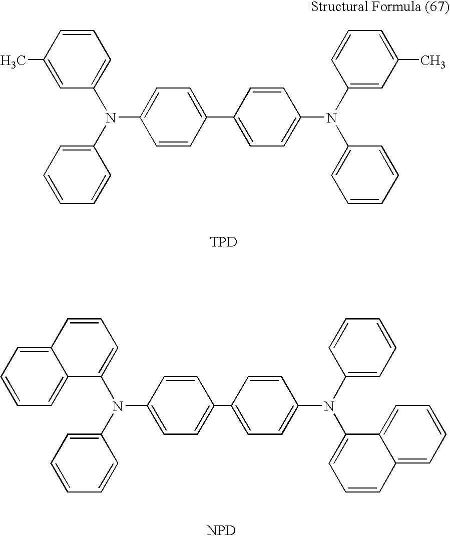

The thickness of the positive hole transport layer may be properly selected depending on the application; preferably, the thickness is 1 nm to 500 nm, more preferably 10 nm to 100 nm.

The positive hole transport layer may be properly formed by, for example, vapor deposition processes, wet film forming processes, electron beam processes, sputtering processes, reactant sputtering processes, MBE (molecular beam epitaxy) processes, cluster ion beam processes, ion plating processes, plasma polymerization processes (high frequency excitation ion plating processes), molecule laminating processes, LB processes, printing processes and transfer processes.

Positive Hole Blocking Layer

The positive hole blocking layer may be properly selected depending on the application, preferably, from those capable of blocking positive holes injected from the positive electrode. The material of the blocking layer may be properly selected depending on the purpose.

In the construction where the organic EL element involves the positive hole blocking layer, the positive holes transported from the side of the positive electrode can be blocked by the positive hole blocking layer; on the other hand, electrons transported from the negative electrode can pass through the positive hole blocking layer to reach the light emitting layer. Consequently, the electrons and the positive holes can recombine efficiently at the light emitting layer, thus the recombination of the electrons and the positive holes can be hindered at the organic thin layers other than the light emitting layer, the effective light emission and the color purity can be advantageously taken from the intended luminescent material.

The positive hole blocking layer is preferably disposed between the light emitting layer and the electron transport layer. The thickness of the positive hole blocking layer may be properly selected depending on the purpose; for example, the thickness is 1 nm to 500 nm, preferably 10 nm to 50 nm.

The positive hole blocking layer may be of single layer structure or laminate structure.

The positive hole blocking layer may be properly formed by, for example, vapor deposition processes, wet film forming processes, electron beam processes, sputtering processes, reactant sputtering processes, MBE (molecular beam epitaxy) processes, cluster ion beam processes, ion plating processes, plasma polymerization processes (high frequency excitation ion plating processes), molecule laminating processes, LB processes, printing processes and transfer processes.

Electron Transport Layer

The electron transport layer may be properly selected depending on the application, preferably, from those capable of at least one of transporting electrons from the negative electrode and blocking the positive holes injected from the positive electrode.

The material of the electron transport layer may be properly selected depending on the purpose; examples thereof include quinoline derivatives such as aluminum quinoline complexes (Alq), oxadiazole derivatives, triazole derivatives, phenanthroline derivatives, perylene derivatives, pyridine derivatives, pyrimidine derivatives, quinoxaline derivatives, diphenylquinone derivatives, nitro-substituted fluorophene derivatives, and the like.

In the processes where the materials of the electron transport layer and the materials of the light emitting layer are combined then to form a film, an electron transport layer/light emitting layer can be formed; in the processes where the materials of the positive hole transport layer is further combined then to form a film, an electron transport layer/positive hole transport layer/light emitting layer can be formed. In these processes, polymers may be utilized together with, such as poly(vinylcarbazole) and polycarbonate.

The thickness of the electron transport layer may be properly selected depending on the purpose; for example, the thickness is usually 1 nm to 500 nm, preferably 10 nm to 50 nm. The electron transport layer may be of single layer structure or laminate structure.

It is preferred that the electron transporting material utilized for the electron transport layer adjacent to the light emitting layer has an optical absorption edge of which the wavelength is shorter than that of the organometallic complex, from the viewpoint that the light emitting region in the organic EL element is confined to the light emitting layer and needless luminescence is excluded from the electron transport layer. Examples of the electron transporting material, having an optical absorption edge of which the wavelength is shorter than that of the organometallic complex, include phenanthroline derivatives, oxadiazole derivatives, triazole derivatives, 2,9-dimethyl-4,7-diphenyl-1,10-phenanthroline expressed by the structural formula (68) and the compounds shown below:

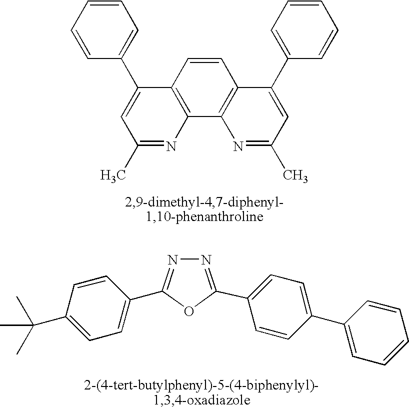

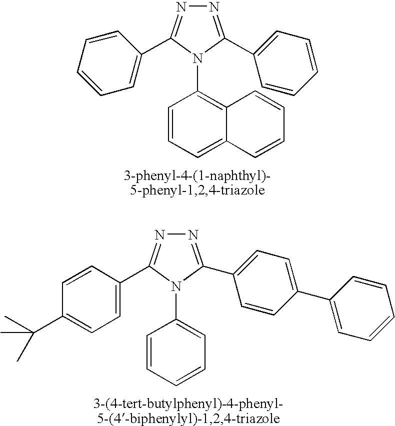

The electron transport layer may be properly formed by, for example, vapor deposition processes, wet film forming processes, electron beam processes, sputtering processes, reactant sputtering processes, MBE (molecular beam epitaxy) processes, cluster ion beam processes, ion plating processes, plasma polymerization processes (high frequency excitation ion plating processes), molecule laminating processes, LB processes, printing processes and transfer processes.

Electron Injection Layer

The material of the electron injection layer may be properly selected depending on the purpose; preferable examples are alkaline metal fluorides such as lithium fluoride, alkaline earth metal fluorides such as strontium fluoride etc. The thickness of the electron injection layer may be properly selected depending on the purpose; the thickness is typically 0.1 nm to 10 nm, preferably 0.5 nm to 2 nm. The electron injection layer may be properly formed by, for example, vapor deposition processes, electron beam processes, sputtering processes, reactant sputtering etc.

Other Layers

The organic EL element according to the present invention may contain other layers selected properly depending on the purpose; preferable examples are a color transfer layer, a protective layer etc.

Color Transfer Layer

It is preferred that the color transfer layer contains a phosphorescent material, more preferable the organometallic complex according to the present invention. The color transfer layer may be formed of the organometallic complex itself, or may contain the other optional materials.

The organometallic complex in the color transfer layer may be used singly or in combination of two or more.

In general, it is well-known that the wavelength of lights for exciting organic molecules and the wavelength of lights emitted from the organic molecules are different since the organic molecules lose a part of the excitation energy due to intramolecular and/or intermolecular effects in a non-radiation form such as thermal energy before the turn into a ground state while emitting a light. The energy difference of the excitation light and emission light is called as Stokes shift. Conventionally, the color transfer materials used in the color transfer layers have been fluorescent materials, of which the emission light is only from the singlet state, considering the margin of selective materials. However, the fluorescent materials have typically a small Stokes shift (<100 nm) such that the emission, corresponding to the strongest absorption band in visible range, appears at the wavelength inconsiderably longer than that of the absorption wavelength, therefore, it is impossible to absorb a blue light and transform to into a red light for example. On the other hand, the organometallic complex of the present invention is a phosphorescent material, therefore, when excited by a light with certain wavelength and a singlet excited state is formed, a transition can progress rapidly into a lower energy state of triplet excited state to emit phosphorescence, thus the Stokes shift is larger than that of fluorescent materials (in conventional organic compounds, the energy of triplet excited state is 0.1 eV to 2 eV lower than that of singlet excited state). For example, concerning the applications where an emission of an original blue color is transferred into a red color, the color transfer efficiency per molecule may be relatively high, since the color transforming layer with phosphorescent materials can exhibit higher absorption efficiencies compared to those with fluorescent materials. In other words, since the color transfer layer with the fluorescent materials does not absorb blue light, more blue light transmits through the color transfer layer. For the countermeasure, it may be possible to increase the blue light absorption and to enhance the red light by thickening the color transfer layer without changing the dispersion concentration. However, such countermeasure tends to suffer from serious problems such as deterioration of materials of the organic EL elements and the related occurrences of non-emitting regions by action of exudates, e.g. moisture and residual organic solvents, from the color transfer layers upon producing the organic EL elements; accordingly, it is preferred that the color transfer layer is as thin as possible. Furthermore, in the color transfer layers with fluorescent materials, the lower absorption efficiencies of guest materials can be compensated by combining host materials that absorbs a blue light; however, such host materials are not necessarily required and higher color transfer efficiencies may be obtained alone in the color transfer layers with phosphorescent materials. Accordingly, it is advantageous that many problems such as the concerned light emission from the host molecule, or deteriorating forming property of color transfer layer, or cost for making the plate in the color transfer layer formed by combination of host, may be solved simultaneously. Furthermore, in cases where host materials are employed, the fluorescent materials often reduce significantly the light emission due to concentration quenching under higher concentrations, meanwhile the phosphorescent materials have been found that the concentration quenching is less likely to occur compared to the fluorescent materials and the dispersed concentration is substantially non-limited. For example, for the phosphorescent materials, even if they are powder state, those emit light are more than fluorescent materials, conversely, when dispersion concentration is very low, due to the optical quenching effect of oxygen molecule, light emission is weaken. In powder state, the effectiveness of the case utilizing phosphorescent materials is the point where suppressed deterioration of color transfer layer can be achieved. Color transfer layer is always exposed to light during the plate-forming state such as photolithography or ITO patterning process where color transfer is carried out as an element, therefore, the declining color transfer efficiency by photo-deterioration becomes a problem. In the case of using luminous material dispersed in color transfer layer, as luminous material per unit is exposed to light, the deterioration is very fast and it is very difficult to prevent it. As compared with this, as color transfer layer using powder state phosphorescent material is exposed to light in bulk, color transfer layer of suppressed deterioration, long lifetime and unchangeable transformation efficiency may be obtained.

The position, where the color transfer layer is disposed, may be properly selected depending on the application; preferably, the color transfer layer is disposed on picture elements in cases of full-color displays.

It is preferred for the organic EL elements that the color transfer layer can transfer the incident light to a light of which the wavelength is no less than 100 nm longer than the incident light, more preferably no less than 150 nm longer than the incident light.

It is preferred that the color transfer layer can change the lights between the wavelength region of violet to blue into a red light. The process for forming the color transfer layer may be properly selected depending on the purpose; examples thereof include vapor-deposition processes, coating processes etc.

In the present invention, conventional color filters may also be utilized for the color transfer layer.

The protective layer may be properly selected depending on the purpose, for example, from those capable of preventing the molecules or substances which promote deterioration of the organic EL elements, such as moisture and oxygen, from penetrating into the organic EL elements.

Examples of the material of the protective layer include metals such as In, Sn, Pb, Au, Cu, Ag, Al, Ti and Ni; metal oxides such as MgO, SiO, SiO₂, Al₂O₃, GeO, NiO, CaO, BaO, Fe₂O₃, Y₂O₃ and TiO₂; nitrides such as SiN and SiNₓO_{y}; metal fluorides such as MgF₂, LiF, AlF₃ and CaF₂; polyethylene, polypropylene, polymethyl methacrylate, polyimide, polyurea, polytetrafluoroethylene, polychlorotrifluoroethylene, polydichlorodifluoroethylene, copolymers of chlorotrifluoroethylene and dichlorodifluoroethylene; copolymers by copolymerizing a monomer mixture containing tetrafluoroethylene and at least one comonomer; fluorine-containing copolymers having a ring structure in the copolymer main chain, water-absorbing substances having a water absorption rate of 1% or more, and dampproof substances having a water absorption rate of 0.1% or less.

The protective layer may be properly formed by, for example, vapor deposition processes, wet film forming processes, sputtering processes, reactant sputtering processes, MBE (molecular beam epitaxy) processes, cluster ion beam processes, ion plating processes, plasma polymerization processes (high frequency excitation ion plating processes), molecule laminating processes, LB processes, printing processes and transfer processes.

Layer Construction

The layer construction of the organic EL element according to the present invention may be properly selected depending on the purpose; preferable examples of the layer structure are (1) to (13) shown below:

(1) positive electrode/positive hole injection layer/positive hole transport layer/light emitting layer/electron transport layer/electron injection layer/negative electrode;

(2) positive electrode/positive hole injection layer/positive hole transport layer/light emitting layer/electron transport layer/negative electrode;

(3) positive electrode/positive hole transport layer/light emitting layer/electron transport layer/electron injection layer/negative electrode;

(4) positive electrode/positive hole transport layer/light emitting layer/electron transport layer/negative electrode;

(5) positive electrode/positive hole injection layer/positive hole transport layer/light emitting and layer-electron transport layer/electron injection layer/negative electrode;

(6) positive electrode/positive hole injection layer/positive hole transport layer/light emitting and electron transport layer/negative electrode;

(7) positive electrode/positive hole transport layer/light emitting and electron transport layer/electron injection layer/negative electrode;

(8) positive electrode/positive hole transport layer/light emitting and electron transport layer/negative electrode;

(9) positive electrode/positive hole injection layer/positive hole transport and light emitting layer/electron transport layer/electron injection layer/negative electrode;

(10) positive electrode/positive hole injection layer/positive hole transport and light emitting layer/electron transport layer/negative electrode;

(11) positive electrode/positive hole transport and light emitting layer/electron transport layer/electron injection layer/negative electrode;

(12) positive electrode/positive hole transport and light emitting layer/electron transport layer/negative electrode; and

(13) positive electrode/positive hole transport and light emitting and electron transport layer/negative electrode, and the like.

In cases where the organic EL elements contain a positive hole blocking layer, the embodiments (1) to (13) described above may preferably have a layer construction in which the positive hole blocking layer is interposed between the light emitting layer and electron transport layer.

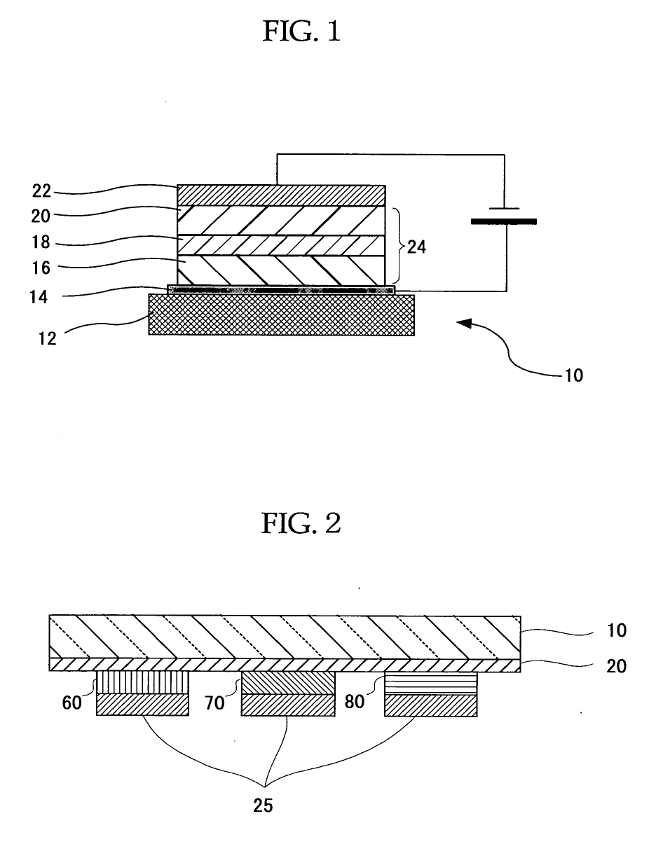

Among these layer constructions, FIG. 1 shows the embodiment (4) of positive electrode/positive hole transport layer/light emitting layer/electron transport layer/negative electrode. The organic EL element 10 has a layer construction having positive electrode 14 (e.g. ITO electrode) formed on glass substrate 12, positive hole transport layer 16, light emitting layer 18, electron transport layer 20, and negative electrode 22 (e.g. Al-Li electrode) laminated in this order. The positive electrode 14 (e.g. ITO electrode) and the negative electrode 22 (e.g. Al-Li electrode) are interconnected through the power supply. Organic thin film layer 24 is formed from the positive hole transport layer 16, light emitting layer 18 and electron transport layer 20.

It is preferred that the luminance half-life of the organic EL elements according to the present invention is as long as possible; for example, the half-life is preferably 20 hours or longer, more preferably 40 hours or longer, particularly preferably 60 hours or longer in a continuous drive at current density of 50 A/m².

The emitting peak wavelength of the organic EL elements according to the present invention may be properly selected from the visible light range, for example, 600 nm to 650 nm is preferable.

It is preferred that the organic EL elements according to the present invention emit a light at an emission voltage of 10 V or less, more preferably 8 V or less, still more preferably 7 V or less.

The current efficiency of the organic EL elements according to the present invention is preferably 10 cd/A or more at a current density of 5 A/m², more preferably 30 cd/A or more, still more preferably 40 cd/A or more.

The organic EL elements according to the present invention may be successfully applied in various fields, such as for computers, display devices in vehicles, field display devices, home apparatuses, industrial apparatuses, household electric appliances, traffic display devices, clock display devices, calendar display units, luminescent screens and audio equipment; and are particularly suitable for lighting systems and the organic EL displays of the present invention described below.

Organic EL Display

The organic EL displays according to the present invention may be constructed in accordance with conventional manners except that the organic EL elements according to the present invention are employed. The organic EL displays may be of monochrome light, multi-color light, or a full color type.

The organic EL displays may be formed into a full color type by methods introduced in “Japan Display Monthly, September 2000, pp. 33-37”, that is, a method for emitting lights in three colors in which the light emitting organic EL elements respectively corresponding to the three primary colors (blue (B), green (G), red (R)) are disposed on a substrate, the white method wherein the white light from an organic EL element for white light emission is divided into the three primary colors by color filters, and the color conversion method wherein a blue light emitted by an organic EL element which emits blue light is converted into red (R) and green (G) by a fluorescent pigment layer. In the present invention, since the organic EL element of the invention emits a red light, the three color light emitting method and color conversion method can be used, the three color light emitting method being particularly suitable.

In cases where the inventive organometallic complexes are employed as a color transfer material, the color transfer method etc. described above can be properly used in particular.

The specific examples of organic EL display of the present invention on the basis of the color transfer method, for example, the organic EL display as shown in FIG. 2, have an organic thin film layer 30 for blue light emission arranged on the whole surface of an electrode 25 situated corresponding to the pixel, and further on this layer, a transparent electrode 20. On the transparent electrode 20, color transfer layer 60 for red light emission and laminate of red color filter 65, and color transfer layer 70 for green light emission and laminate of green color filter 80 are situated through a protecting layer (flattened layer) 15; and over these, a glass plate 10 is arranged.

When a voltage is applied between the electrode 25 and the transparent electrode 20 in this organic EL display, the organic thin film layer 30 for blue light emission emits a blue light. One part of this blue light emission transmits through the transparent electrode 20, transmits through the protecting layer 15 and the glass plate 10 and exits outside. On the other hand, in the area where the color transfer layer 60 for red light emission and the color transfer layer 70 for green light emission exist, the blue light emission is converted to red light and green light, respectively, in these color transfer layers and further by transmitting through red color filter 65 and green color filter 80, they become red light emission and green light emission, respectively, and transmit through the glass plate 10. As a result, this organic EL can display in full color.

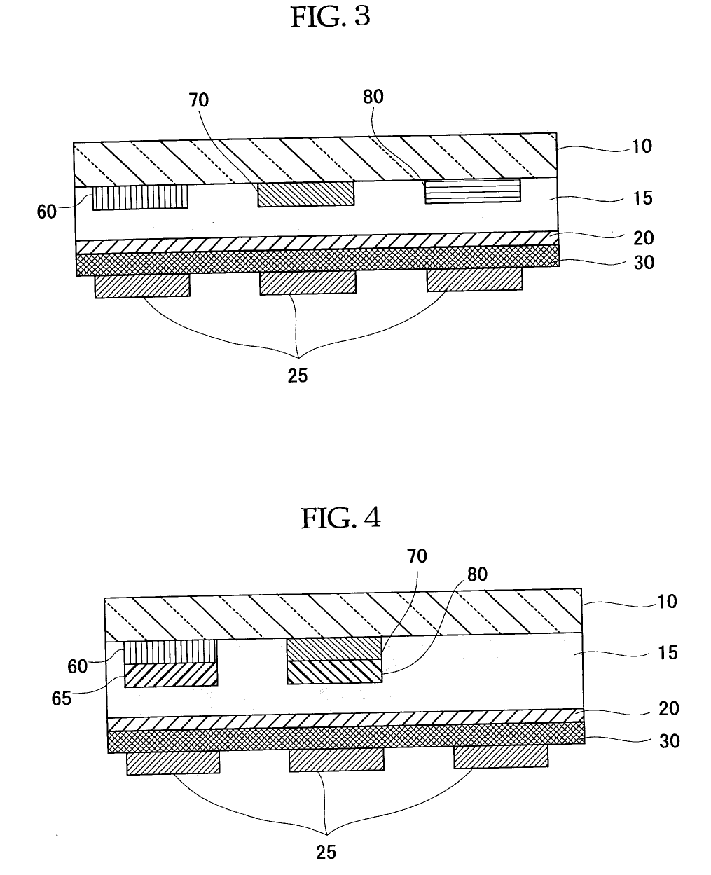

In the case where the color transfer layers 60 and 70 are formed of an organometallic complex (phosphorescent material) according to the present invention, the organometallic complex itself can be formed into a film without combining with the host material in the color transfer layer for red emission, which can bring about easy production and higher color transfer efficiency. FIG. 3 shows an exemplary construction of an organic EL display on the basis of three colors emitting method, and FIG. 4 shows an exemplary construction of an organic EL display on the basis of the white method. The reference numbers in FIGS. 3 and 4 indicate the same ones as those in FIG. 2.

In the production of the full color organic EL display on the basis of the three color emitting method, for example, when the organic EL element of the present invention is used for red light emission (the organic EL element of the present invention may be used for light emission of other colors, and also all the colors may be formed by the organic EL element of the present invention), an organic EL element for green light emission and an organic EL element for blue light emission are further required.

The organic EL element for the blue light emission may be properly selected from conventional ones, for example, from those having a layer construction of ITO (positive electrode)/NPD described above/Al—Li (negative electrode) etc.

The organic EL element for green light emission may be properly selected from conventional ones, for example, from those having a layer construction of ITO (positive electrode)/NPD described above/Alq described above/AL-Li (negative electrode).

The organic EL display may be properly selected depending on the purpose; preferable examples thereof include the passive matrix panel and active matrix panel shown in “Nikkei Electronics, No. 765, Mar. 13, 2000, pp. 55-62”.

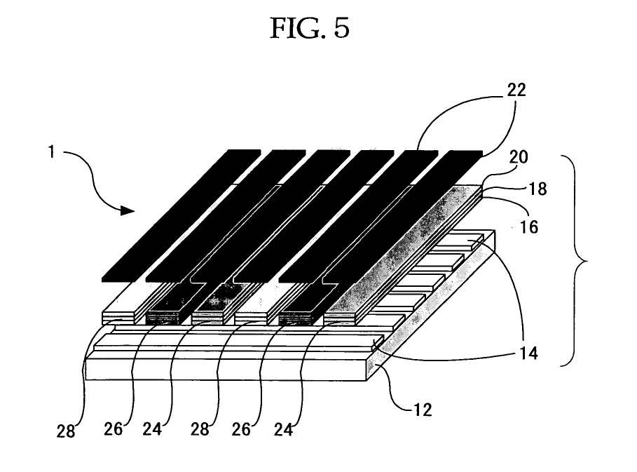

The passive matrix panel, as shown in FIG. 5, has belt-like positive electrodes 14 (e.g. ITO electrodes) arranged in parallel each other on a glass substrate 12; belt-like organic thin film layer 24 for red light emission, organic thin film layer 26 for blue light emission and organic thin film layer 28 for green light emission are arranged sequentially in parallel and approximately perpendicular to the positive electrode 14 on the positive electrode 14; and negative electrodes 22 of identical shape with and on the organic thin film layer 24 for red light emission, the organic thin film layer 26 for blue light emission, and the organic thin film layer 28 for green light emission.

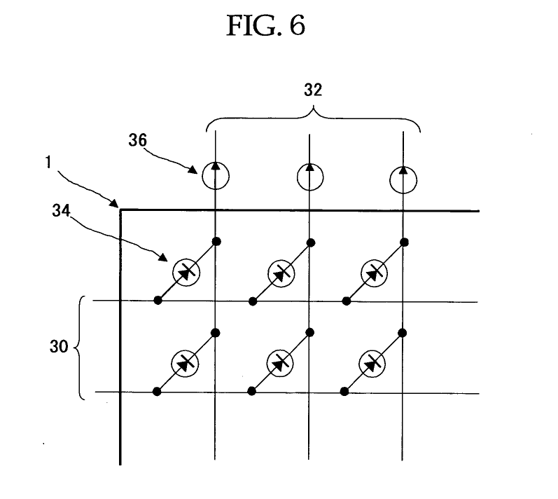

In the passive matrix panel, positive electrode lines 30 consisting of plural positive electrodes 14, and negative electrode lines 32 consisting of plural negative electrodes 22, for example, intersect approximately at right angles to form a circuit, as shown in FIG. 6. Each of the organic thin film layers 24, 26, 28 for red light emission, blue light emission and green light emission situated at each intersection point functions as a pixel, there being plural organic EL elements 34 corresponding to each pixel. In this passive matrix panel, when a current is applied by a constant current source 36 to one of the positive electrodes 14 in the positive electrode lines 30, and one of the negative electrodes 22 in the negative electrode lines 32, a current is applied to the organic EL thin film layer situated at the intersection, and the organic EL thin film layer at this position emits a light. By controlling the light emission of this pixel unit, a full color picture can easily be formed.

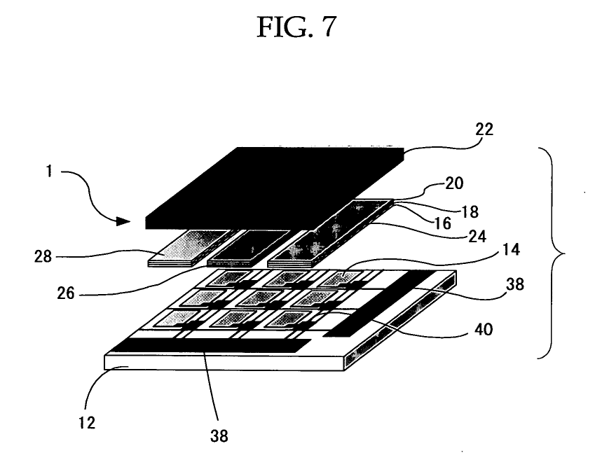

In the active matrix panel, for example, scanning lines, data lines and current supply lines are arranged in a grid pattern on the glass substrate 12, as shown in FIG. 7. TFT circuit 40 connected by the scanning lines forming the grid pattern is disposed in each square, and positive electrode 14 (e.g. ITO electrode) disposed in each square can be driven by the TFT circuit 40. The belt-like organic thin film layer 24 for red light emission, organic thin film layer 26 for blue light emission and organic thin film layer 28 for green light emission are arranged sequentially in parallel. The negative electrodes 22 are also arranged so as to cover the organic thin film layer 24 for red light emission, organic thin film layer 26 for blue light emission and organic thin film layer 28 for green light emission. The organic thin film layer 24 for red light emission, organic thin film layer 26 for blue light emission and organic thin film layer 28 for green light emission respectively form a positive hole transport layer 16, light emitting layer 18 and electron transport layer 20.

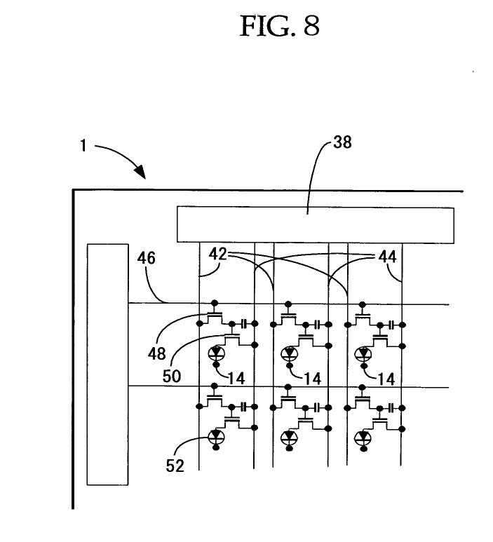

In the active matrix panel, plural scanning lines 46 parallel to each other, plural data lines 42 parallel to each other and current supply lines 44 intersect approximately at right angles to form squares, as shown in FIG. 8, and switching TFT 48 and drive TFT 50 are connected to each square to form a circuit. When an electric current is applied from drive circuit 38, the switching TFT 48 and drive TFT 50 can be driven for each square. In each square, the organic thin film elements 24, 26, 28 for blue light emission, green light emission and red light emission function as a pixel. In this active matrix panel, when a current is applied from the drive circuit 38 to one of the scanning lines 46 arranged in the horizontal direction, and the current supply line 44 arranged in the vertical direction, the switching TFT 48 situated at the intersection is driven, the drive TFT 50 is driven as a result, and the organic EL element 52 at this position emits light. By controlling the light emission of this pixel unit, a full color picture can easily be formed.

The organic EL displays according to the present invention may be suitably used in various applications such as televisions, cellular phones, computers, display devices in vehicles, field display devices, home apparatuses, industrial apparatuses, household electric appliances, traffic display devices, clock display devices, calendar display units, luminescent screens and audio equipment.

The present invention will be explained with reference to non-limiting examples of the present invention.

### SYNTHESIS EXAMPLE 1

### Synthesis of Pt(3,5-di(2-pyridyl)toluene)biphenyloxide

Pt(3,5-di(2-pyridyl)toluene)(biphenyloxide) (hereinafter referred to as “Pt(dpt)(obp)”) was synthesized as follows. Specifically, 3,5-dibromotoluene (5.0 g; 20 mmol), 2-tri-n-butylstannylpyridine (26.9 g, 73 mmol), bis(triphenylphosphine)palladium dichloride (1.55 g, 2.2 mmol), and lithium chloride (11.7 g, 276 mmol) were added to 130 ml of toluene, and the mixture was refluxed for two days. After allowing the mixture to cool, 50 ml of saturated KF aqueous solution was added. The deposited solid was collected by filtering, the solid was washed using a small amount of cooled toluene (200 ml by 3 times) then dried under vacuum. The resulting solid was rinsed sufficiently in a mixture solution of dichloromethane and NaHCO₃. The organic layer of the liquid was separated, which was dried over MgSO₄ powder, then solvents were removed from the solid using an evaporator. The solid was recrystallized using dichloromethane to obtain an intended gray solid of 3,5-di(2-pyridyl)toluene in an amount of 2.2 g. The yield was 45%.

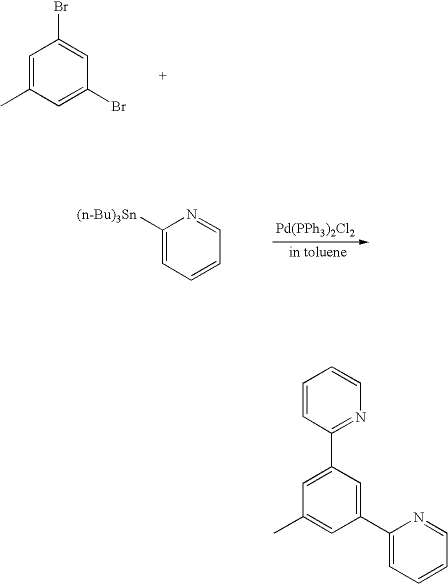

Then the resulting 3,5-di(2-pyridyl)toluene (300 mg, 1.2 mmol) and K₂PtCl₄ (550 mg, 1.3 mmol) were added to degassed acetic acid (30 ml), then the mixture was refluxed at 130° C. for two days. The reactant was allowed to cool then the resulting precipitate of light yellow crystal was filtered and collected. The collected solid was washed sufficiently using methanol, water and dimethylether and dried under vacuum. The resulting coarse powder was recrystallized using dichloromethane to obtain an intended yellow powder of Pt(dpt)Cl in an amount of 436 mg. The yield was 77%.

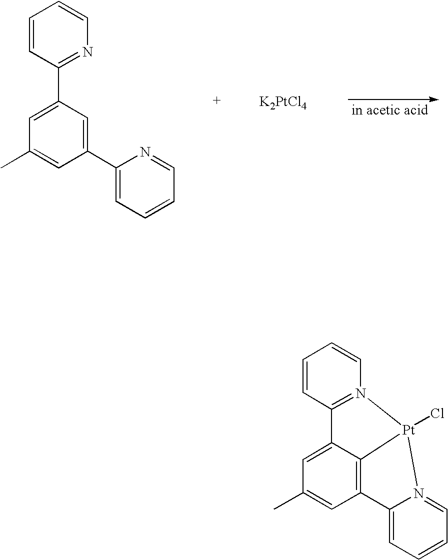

Separately, p-hydroxybiphenyl (2.08 g, 0.01 mol) and KOH (2 g, 0.036 mol) was added to acetone (30 ml) and the mixture was stirred at room temperature for 30 minutes, to which a few drops of water was added thereby KOH was dissolved into and a uniform solution was formed. After the reactant was stirred continuously at room temperature for three hours still further, a white powder of intended product was deposited then to filter it. The resulting solid was washed using acetone, a small amount of methanol and diethylether sequentially, and dried under vacuum, consequently to obtain a white crystalline powder of intended biphenyloxide potassium. The yield was 85%.

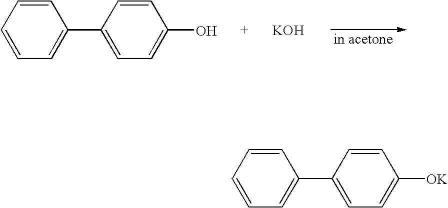

Next, the resulting Pt(dpt)Cl (100 mg, 0.21 mmol) was added to acetone (30 ml) and stirred, to which the biphenyloxide potassium (66 mg, 0.32 mmol) described above dissolved in methanol (20 ml) was added slowly dropwise and stirred at room temperature for 10 minutes. The reactant was stirred for three hours while heating since addition of a few drops of pure water progresses the reaction and a pale yellow solid initiates its deposition. The reactant was allowed to cool then the deposition of pale yellow solid was separated by filtration, washed sufficiently using pure water, methanol and diethylether in order, and dried under vacuum to obtain a pale yellow solid of Pt(dpt)(obp). The yield was 77%. FIG. 9 shows the IR spectrum of the Pt(dpt)(obp).

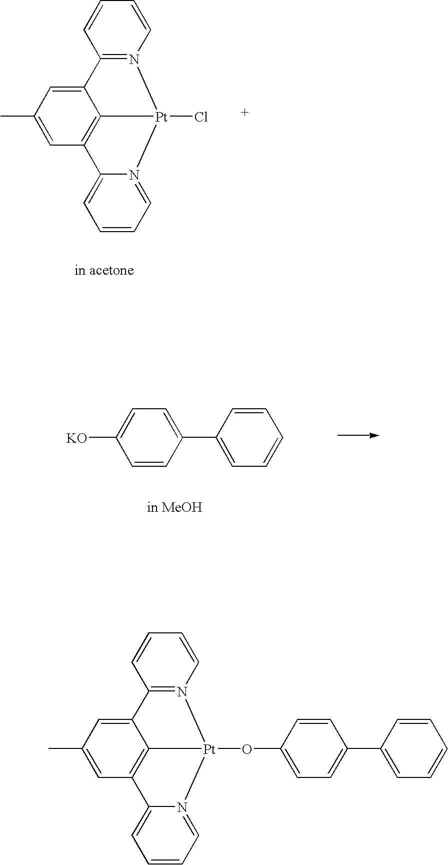

### SYNTHESIS EXAMPLE 2

### Synthesis of Pt(3,5-di(2-pyridyl)toluene)(OH) (hereinafter referred to as “Pt(dpt)(OH)”)

Pt(dpt)Cl was prepared in the same manner as Synthesis Example 1, then the resulting Pt(dpt)Cl (100 mg, 0.21 mmol) was added to acetone (30 ml) and stirred, to which KOH powder (56 mg, 1 mmol) was added to stir at room temperature for 10 minutes. Addition of a few drops of pure water initiated deposition of a yellow solid. The reactant was stirred for three hours while heating and allowed to cool then the deposited solid was separated by filtration, washed sufficiently using pure water, methanol and diethylether in order, and dried under vacuum to obtain a yellow solid of Pt(dpt)(OH). The yield was 68%. FIG. 10 shows the IR spectrum of the Pt(dpt)(OH).

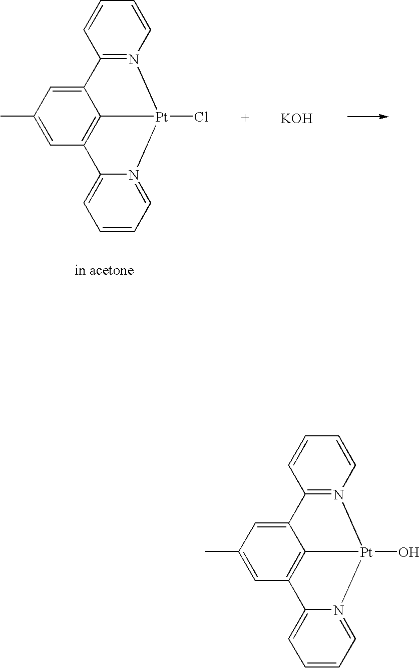

### SYNTHESIS EXAMPLE 3

### Synthesis of Pt(3,5-di(2-pyridyl)toluene)(1,2,4-triazolate) (hereinafter referred to as “Pt(dpt)(taz)”)

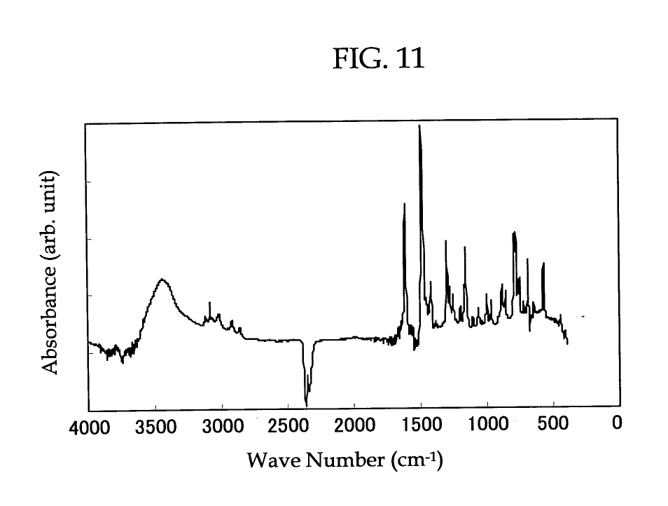

Pt(dpt)Cl was prepared in the same manner as Synthesis Example 1, then the resulting Pt(dpt)Cl (100 mg, 0.21 mmol) was added to acetone (30 ml) and stirred, to which 1,2,4-triazole sodium salt (29 mg, 0.32 mmol) was added to stir at room temperature for 10 minutes. Addition of a few drops of pure water initiated deposition of a yellow solid. The reactant was stirred for three hours while heating and allowed to cool then the deposited solid was separated by filtration, washed sufficiently using pure water, methanol and diethylether in order, and dried under vacuum to obtain a yellow solid of Pt(dpt)(taz). The yield was 82%. FIG. 11 shows the IR spectrum of the Pt(dpt)(taz).

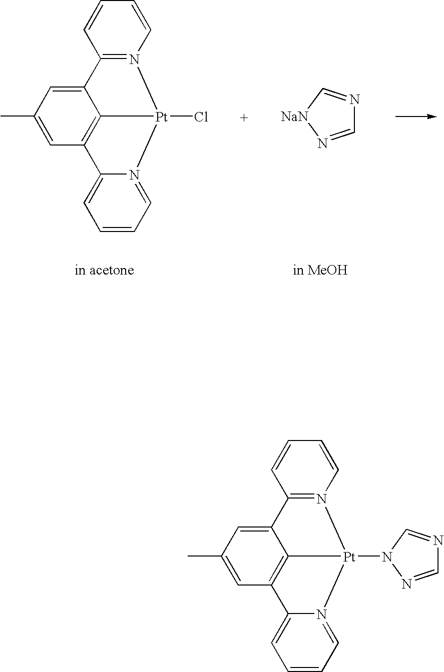

### SYNTHESIS EXAMPLE 4

### Synthesis of Pt(3,5-di(2-pyridyl)toluene)(benzothiazole-2-thiolate) (hereinafter referred to as “Pt(dpt)(sbtz)”)

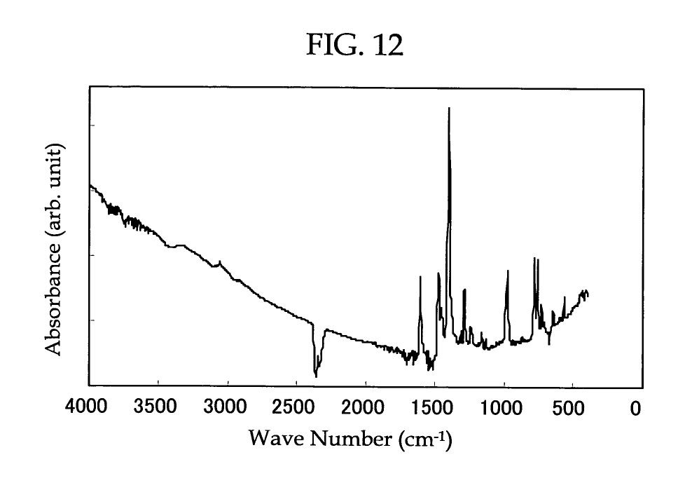

Pt(dpt)Cl was prepared in the same manner as Synthesis Example 1, then the resulting Pt(dpt)Cl (100 mg, 0.21 mmol), 2-mercaptobenzothiazole (42.1 mg, 0.25 mmol), and DMSO (30 ml) were charged into a three-neck flask of 100 ml, and stirred under nitrogen atmosphere. NaOH powder (200 mg, 5 mmol) was added to the mixture, and the mixture was refluxed for five hours then allowed to cool. A large amount of water was added to the reactant; consequently, the solution changed its color from yellow to red then from red to brown, and a solid of yellow and/or brown was deposited. The reactant was stirred at room temperature for two hours additionally. The deposited yellow solid was separated by filtration, washed sufficiently using pure water, acetone and diethylether in order, and dried under vacuum to obtain a yellow solid of Pt(dpt)(sbtz). The yield was 35%. FIG. 12 shows the IR spectrum of the Pt(dpt)(sbtz).

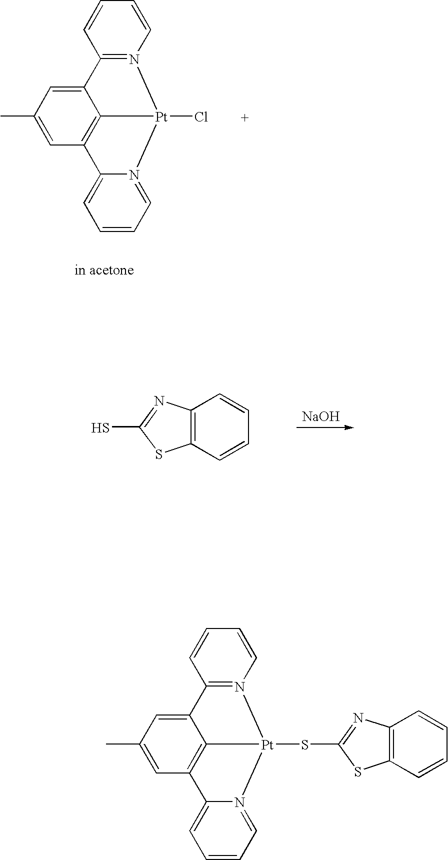

### SYNTHESIS EXAMPLE 5

### Synthesis of Pt(3,5-di(1-isoquinolyl)toluene)(biphenyloxide) (hereinafter referred to as “Pt(diqt)(obp)”)

3,5-di(1-isoquinolyl)toluene was synthesized in the same manner as the synthesis of 3,5-di(2-pyridyl)toluene in Synthesis Example 1 except that 2-tri-n-butylstannylpyridine was changed into 1-tri-n-butylstannylisoquinoline. The yield was 54%. In addition, Pt(3,5-di(1-isoquinolyl)toluene)Cl (hereinafter referred to as “Pt(diqt)Cl”) was synthesized in the same manner as the synthesis of Pt(dpt)Cl except that 3,5-di(2-pyridyl)toluene was changed into 3,5-di(1-isoquinolyl)toluene. The yield was 42%. In addition, Pt(diqt)(obp) was synthesized in the same manner as that of the Pt(dpt)(obp) except that Pt(dpt)Cl was changed into Pt(diqt)Cl; consequently, an orange powder of Pt(diqt)(obp) was obtained. The yield was 83%.

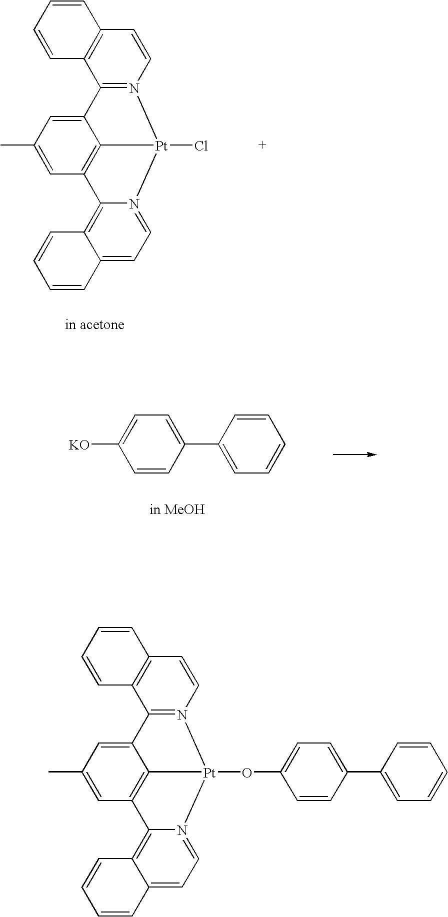

### SYNTHESIS EXAMPLE 6

### Synthesis of Pt(3,5-di(1-isoquinolyl)toluene)(OH) (hereinafter referred to as “Pt(diqt)(OH)”)

Pt(3,5-di(1-isoquinolyl)toluene)Cl (hereinafter referred to as “Pt(diqt)Cl”) was prepared in the same manner as Synthesis Example 5. Then the resulting Pt(diqt)Cl (100 mg, 0.21 mmol) was added to acetone (30 ml) and stirred, to which KOH powder (56 mg, 1 mmol) was added to stir at room temperature for 10 minutes. Addition of a few drops of pure water initiated deposition of a yellow solid. The reactant was stirred for three hours while heating and allowed to cool then the deposited solid was separated by filtration, washed sufficiently using pure water, methanol and diethylether in order, and dried under vacuum to obtain an orange powder of Pt(diqt)(OH). The yield was 68%.

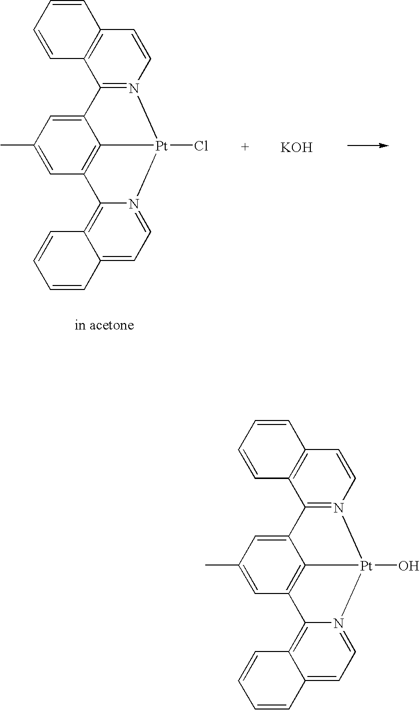

### SYNTHESIS EXAMPLE 7

### Synthesis of Pt(3,5-di(1-isoquinolyl)toluene)(1,2,4-triazolate) (hereinafter referred to as “Pt(diqt)(taz)”)

Pt(3,5-di(1-isoquinolyl)toluene)Cl (hereinafter referred to as “Pt(diqt)Cl”) was prepared in the same manner as Synthesis Example 5. The resulting Pt(diqt)Cl (100 mg, 0.21 mmol) was added to acetone (30 ml) and stirred, to which 1,2,4-triazole sodium salt (29 mg, 0.32 mmol) was added to stir at room temperature for 10 minutes. Addition of a few drops of pure water initiated deposition of a yellow solid. The reactant was stirred for three hours while heating and allowed to cool then the deposited solid was separated by filtration, washed sufficiently using pure water, methanol and diethylether in order, and dried under vacuum to obtain a yellow solid of Pt(dpt)(taz). The yield was 79%.

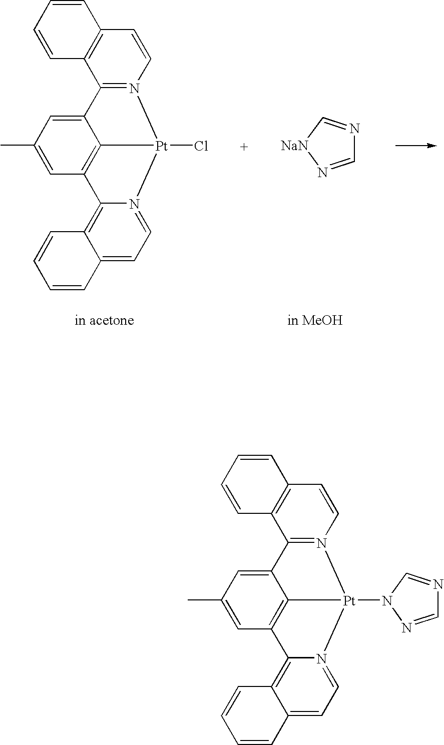

### SYNTHESIS EXAMPLE 8

### Synthesis of Pt(3,5-di(1-isoquinolyl)toluene)(benzothiazole-2-thiolate) (hereinafter referred to as “Pt(diqt)(sbtz)”)

Pt(3,5-di(1-isoquinolyl)toluene)Cl (hereinafter referred to as “Pt(diqt)Cl”) was prepared in the same manner as Synthesis Example 5, then the resulting Pt(diqt)Cl (100 mg, 0.21 mmol), 2-mercaptobenzothiazole (42.1 mg, 0.25 mmol), and DMSO (30 ml) were charged into a three-neck flask of 100 ml, and stirred under nitrogen atmosphere. NaOH powder (200 mg, 5 mmol) was added to the mixture, and the mixture was refluxed for five hours then allowed to cool. A large amount of water was added to the reactant; consequently, the solution changed its color from yellow to red then from red to brown, and a solid of yellow and/or brown was deposited. The reactant was stirred at room temperature for two hours additionally. The deposited yellow solid was separated by filtration, washed sufficiently using pure water, acetone and diethylether in order, and dried under vacuum to obtain an orange powder of Pt(dpt)(sbtz). The yield was 79%.

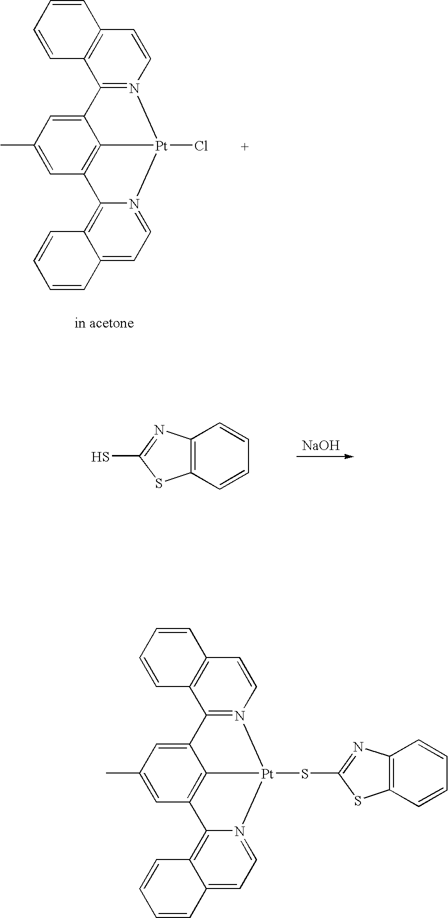

### SYNTHESIS EXAMPLE 9

### Synthesis of Pt(3,5-di(2-pyridyl)pyridine)(biphenyloxide) (hereinafter referred to as “Pt(dppr)(obp)”)

3,5-di(2-pyridyl)pyridine was synthesized in the same manner as the synthesis of 3,5-di(2-pyridyl)toluene in Synthesis Example 1 except that 3,5-bromotoluene was changed into 3,5-bromopyridine. The yield was 54%. In addition, Pt(3,5-di(2-pyridyl)pyridine)Cl (hereinafter referred to as “Pt(dppr)Cl”) was synthesized in the same manner as the synthesis of Pt(dpt)Cl except that 3,5-di(2-pyridyl)toluene was changed into 3,5-di(2-pyridyl)pyridine. The yield was 42%. In addition, Pt(dppr)(obp) was synthesized in the same manner as that of the Pt(dpt)(obp) except that Pt(dpt)Cl was changed into Pt(dppr)Cl; consequently, a pale yellow powder of Pt(dppr)(obp) was obtained. The yield was 65%.

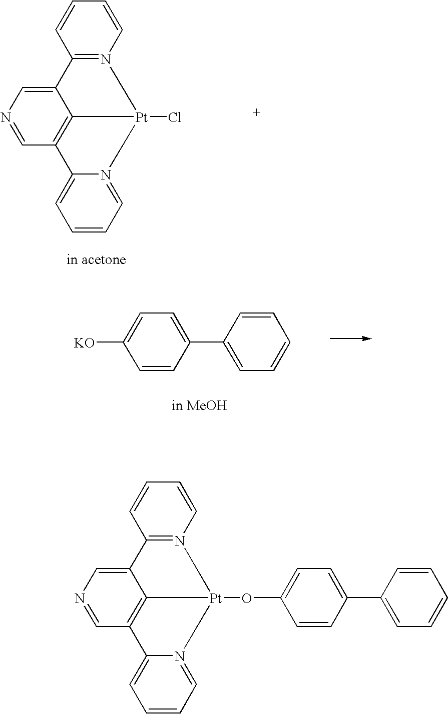

### SYNTHESIS EXAMPLE 10

### Synthesis of Pt(3,5-di(2-pyridyl)pyridine)(OH) (hereinafter referred to as “Pt(dppr)(OH)”)

Pt(3,5-di(2-pyridyl)pyridine)Cl (hereinafter referred to as “Pt(dppr)Cl”) was prepared in the same manner as Synthesis Example 9. Then the resulting Pt(dppr)Cl (100 mg, 0.21 mmol) was added to acetone (30 ml) and stirred, to which KOH powder (56 mg, 1 mmol) was added to stir at room temperature for 10 minutes. Addition of a few drops of pure water initiated deposition of a yellow solid. The reactant was stirred for three hours while heating and allowed to cool then the deposited solid was separated by filtration, washed sufficiently using pure water, methanol and diethylether in order, and dried under vacuum to obtain a pale yellow powder of Pt(dppr)(OH). The yield was 69%.

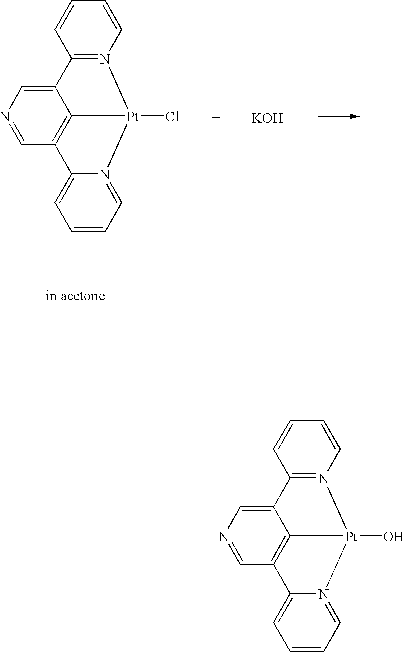

### SYNTHESIS EXAMPLE 11

### Synthesis of Pt(3,5-di(2-pyridyl)pyridine)(1,2,4-triazolate) (hereinafter referred to as “Pt(dppr)(taz)”)

Pt(3,5-di(2-pyridyl)pyridine)Cl (hereinafter referred to as “Pt(dppr)Cl”) was prepared in the same manner as Synthesis Example 9. The resulting Pt(dppr)Cl (100 mg, 0.21 mmol) was added to acetone (30 ml) and stirred, to which 1,2,4-triazole sodium salt (29 mg, 0.32 mmol) was added to stir at room temperature for 10 minutes. Addition of a few drops of pure water initiated deposition of a yellow solid. The reactant was stirred for three hours while heating and allowed to cool then the deposited solid was separated by filtration, washed sufficiently using pure water, methanol and diethylether in order, and dried under vacuum to obtain a pale yellow powder of Pt(dppr)(taz). The yield was 55%.

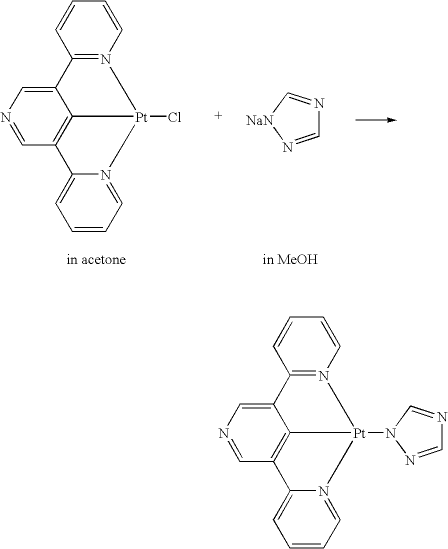

### SYNTHESIS EXAMPLE 12

### Synthesis of Pt(3,5-di(2-pyridyl)pyridine)(benzothiazole-2-thiolate) (hereinafter referred to as “Pt(dppr)(sbtz)”)

Pt(3,5-di(2-pyridyl)pyridine)Cl (hereinafter referred to as “Pt(dppr)Cl”) was prepared in the same manner as Synthesis Example 9, then the resulting Pt(dppr)Cl (100 mg, 0.21 mmol), 2-mercaptobenzothiazole (42.1 mg, 0.25 mmol), and DMSO (30 ml) were charged into a three-neck flask of 100 ml, and stirred under nitrogen atmosphere. NaOH powder (200 mg, 5 mmol) was added to the mixture, and the mixture was refluxed for five hours then allowed to cool. A large amount of water was added to the reactant; consequently, the solution changed its color from yellow to red then from red to brown, and a solid of yellow and/or brown was deposited. The reactant was stirred at room temperature for two hours additionally. The deposited yellow solid was separated by filtration, washed sufficiently using pure water, acetone and diethylether in order, and dried under vacuum to obtain a pale yellow powder of Pt(dppr)(sbtz). The yield was 51%.

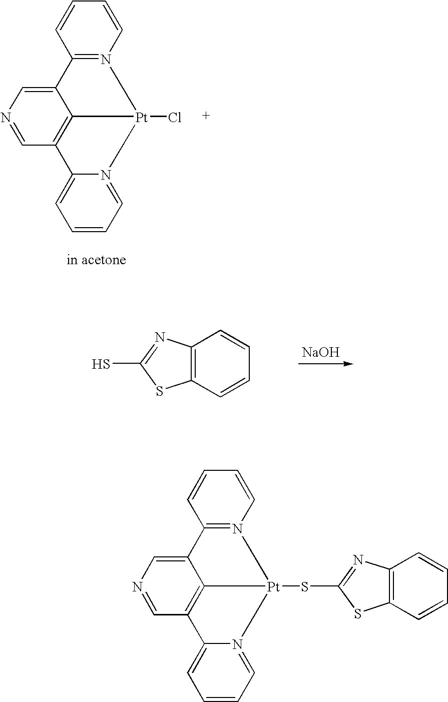

### SYNTHESIS EXAMPLE 13

### Synthesis of Pt(3,5-di(2-pyridyl)toluene)(phenylacetylide) (hereinafter referred to as “Pt(dpt)(acph)”)

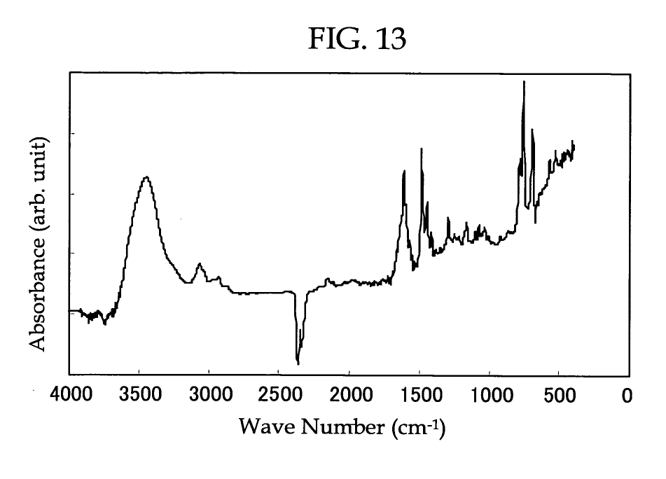

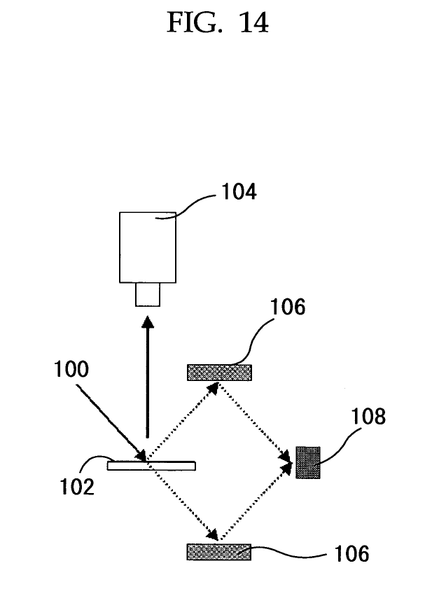

Pt(dpt)Cl was prepared in the same manner as Synthesis Example 1; then the resulting Pt(dpt)Cl (238 mg, 0.5 mmol) and phenylacetylene (153 mg, 1.5 mmol) were mixed to dichloromethane (45 ml), to which then triethylamine (4.5 ml) and CuI (7.5 mg) were added, and the mixture was stirred at room temperature for 24 hours under nitrogen atmosphere. Dichloromethane was distilled away from the reactant liquid, the remaining oily matter was purified by flush chromatography (alumina column, eluate: dichloromethane) thereby to obtain a brownish yellow powder of Pt(dpt)(acph). The yield was 18%. FIG. 13 shows the IR spectrum of Pt(dpt)(acph).

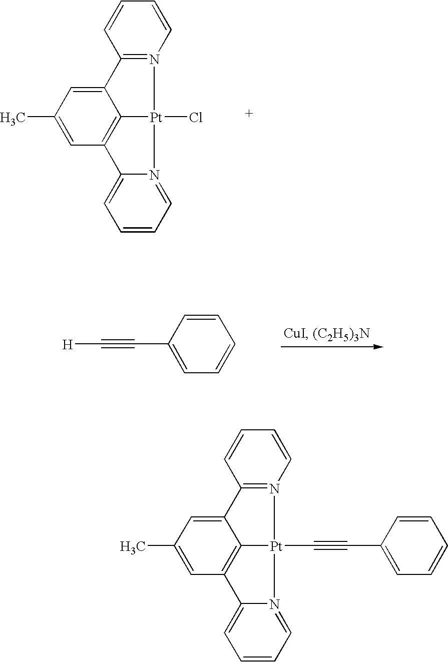

### SYNTHESIS EXAMPLE 14

### Synthesis of Pt(3,5-di(2-pyridyl)toluene)(phenoxide) (hereinafter referred to as “Pt(dpt)(oph)”)

Pt(dpt)Cl (100 mg, 0.21 mmol) was added to acetone (30 ml) and the mixture was stirred, to which a solution of sodium phenoxide 3H₂O (53 mg, 0.32 mmol) in methanol (20 ml) was slowly added drop-wise and the mixture was stirred at room temperature for 10 minutes. Addition of a few drops of pure water promoted the reaction and initiated deposition of a pale yellow solid. The reactant was stirred for three hours while heating followed by allowing to cool then the deposited pale yellow solid was separated by filtration, washed sufficiently using pure water, methanol and diethylether in order, and dried under vacuum to obtain a pale yellow powder of Pt(dpt)(oph). The yield was 80%.

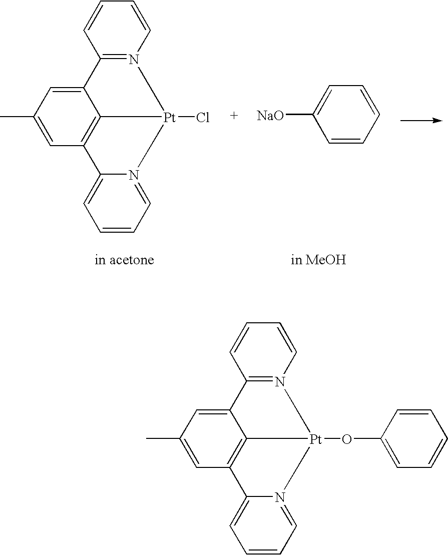

### SYNTHESIS EXAMPLE 15

### Synthesis of Pt(3,5-di(2-pyridyl)toluene)(2-benzothiazole oxalate) (hereinafter referred to as “Pt(dpt)(obtz)”)

Pt(dpt)Cl (100 mg, 0.21 mmol) and 2-hydroxybenzothiazole (47.6 mg, 0.32 mmol) were added to DMSO (30 ml) and the mixture was stirred, to which KOH powder (200 mg, 3.5 mmol) was added and then stirred at room temperature for 10 minutes Addition of a few drops of pure water initiated deposition of a yellow solid. The reactant was stirred for three hours while heating and allowed to cool, then pure water wad added excessively and stirred 30 minutes additionally. The deposited yellow solid was separated by filtration, washed sufficiently using pure water, methanol and diethylether in order, and dried under vacuum to obtain a yellow powder of Pt(dpt)(obtz). The yield was 69%.

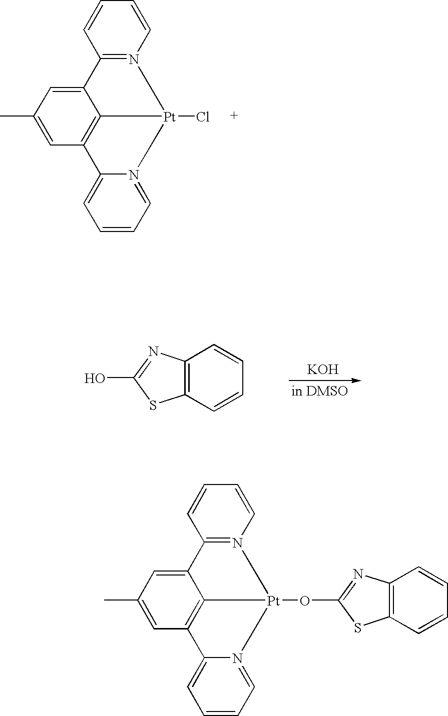

$$( P L quantum efficiency ) = \frac{P ( sample ) / [ I ( substrate ) - I ( sample ) ]}{P ( ref . ) / [ I ( substrate ) - I ( ref . ) ]} \times 22 \%$$

### EXAMPLE 1

Pt(dpt)(obp) and CBP were co-deposited on a quartz substrate to form a thin film (luminescent solid) of 50 nm thick such that 2% of Pt(dpt)(obp) was doped in CBP considering the ratio of vapor-deposition rate. The PL (photoluminescence) quantum yield of the thin film (luminescent solid) was determined as following, using a thin film of an aluminum quinoline having a known PL quantum yield (22%) as the reference.

An excitation light (constant light of 365 nm) from a light source is illuminated slantingly on the thin film on the transparent substrate, and the PL photon number [P(sample)] was calculated by conversing the PL spectrum of the thin film measured by a spectroradiometer (Konica Minolta, CS-1000). At the same time with the measurement of the light emission, the total intensity [I(sample)] of the light, transmitted and reflected from the sample, was detected by a photodiode. Subsequently, the same measurement was also carried out on the Alq3 thin film as the reference to determine the PL photon number [P(ref.)] and total intensity [I(ref.)] of the reflected and transmitted lights. Then the total intensity [I(substrate)] of the reflected and transmitted lights was determined for the transparent substrate itself. The PL quantum yield of thin film sample can be calculated from the following formula.

$$( P L quantum efficiency ) = \frac{P ( sample ) / [ I ( substrate ) - I ( sample ) ]}{P ( ref . ) / [ I ( substrate ) - I ( ref . ) ]} \times 22 \%$$

### EXAMPLES 2 TO 15

The phosphorescence quantum yields of the resulting thin films (luminescent solids) were determined in the same manner with Example 1, except that the organometallic complex Pt(dpt)(obp) of the luminescent material was changed into the organometallic complexes shown in Table 1.

TABLE 1

| | Luminescent | PL Quantum |
|---|---|---|
| | Material | Yield (%) |
| Ex. 1 | Pt(dpt)(obp) | 96 |
| Ex. 2 | Pt(dpt)(OH) | 94 |
| Ex. 3 | Pt(dpt)(taz) | 98 |
| Ex. 4 | Pt(dpt)(sbtz) | 85 |
| Ex. 5 | Pt(diqt)(obp) | 90 |
| Ex. 6 | Pt(diqt)(OH) | 84 |
| Ex. 7 | Pt(diqt)(taz) | 94 |
| Ex. 8 | Pt(diqt)(sbtz) | 80 |
| Ex. 9 | Pt(dppr)(obp) | 95 |
| Ex. 10 | Pt(dppr)(OH) | 90 |
| Ex. 11 | Pt(dppr)(taz) | 96 |
| Ex. 12 | Pt(dppr)(sbtz) | 89 |
| Ex. 13 | Pt(dpt)(acph) | 70 |
| Ex. 14 | Pt(dpt)(oph) | 98 |
| Ex. 15 | Pt(dpt)(optz) | 96 |

The results of Table 1 clearly demonstrate that the phosphorescent thin films using the organometallic complexes according to the present invention represent significantly higher phosphorescent quantum yields.

### EXAMPLE 16

An organic EL element of laminate type was prepared using the resulting organometallic complex Pt(dpt)(obp) as the luminescent material of the light emitting layer. A glass substrate with an ITO electrode was washed using water, acetone and isopropyl alcohol; then 4,4′,4″-tri(2-naphthylphenylamino)triphenylamine (2-TNATA) was formed as a positive hole injection layer on this ITO electrode to 140 nm thick by use of a vacuum vapor deposition apparatus (1×10⁻⁴ Pa, substrate temperature: room temperature). Then, the TPD of 10 nm thick was form on the positive hole injection layer as a positive hole transport layer. On the positive hole transport layer, Pt(dpt)(obp) and CBP were deposited to form a light emitting layer of 30 nm thick such that 2% of Pt(dpt)(obp) was doped in CBP considering the ratio of vapor-deposition rate. The BCP of 20 nm thick was formed as a positive hole blocking layer on the light emitting layer. The Alq of 20 nm thick was formed as an electron transport layer on this positive hole blocking layer. On this electron transport layer, LiF of 0.5 nm thick was then vapor deposited, finally, aluminum of 100 nm thick was vapor deposited, and a sealing was provided under nitrogen atmosphere.

The resulting organic EL element of laminate type was measured in terms of EL properties by applying a voltage between the ITO as a positive electrode and the aluminum electrode as a negative electrode. The voltage, peak wavelength of emission and current efficiency under a current density of 5 A/m² are shown in Table 2.

### EXAMPLES 17 TO 30

Organic EL elements were prepared in the same manner as Example 16 except that the Pt(dpt)(obp) of the luminescent material was changed into the organometallic complexes in Table 2. These organic EL elements were measured in terms of EL properties by applying a voltage between the ITO as a positive electrode and the aluminum electrode as a negative electrode. The voltages, peak wavelengths of emission and current efficiencies under a current density of 5 A/m² are shown in Table 2.

### EXAMPLE 31

An organic EL element was prepared using the resulting organometallic complex Pt(dpt)(obp) as the luminescent material of the light emitting layer. A glass substrate with an ITO electrode was washed using water, acetone and isopropyl alcohol; then poly(3,4-ethylenedioxythiophene): polystyrene sulfonate thin film (PEDOT: PSS thin film) of 50 nm thick was formed as the positive hole injection layer on the ITO by a spin coat process and dried under heating at 200° C. for 2 hours. A light emitting layer of 35 nm of 3% Pt(dpt)(obp) dispersed in polyvinylcarbazole (PVK) was formed on the positive hole injection layer by a spin coating process, and dried at 120° C. for 2 hours. The resulting material was inserted into a vacuum vapor deposition apparatus (1×10⁻⁴ Pa, substrate temperature: room temperature), then the BCP of 20 nm thick was formed as a positive hole blocking layer on the light emitting layer. The Alq of 20 nm thick was formed as an electron transport layer on this positive hole blocking layer. In addition, LiF of 0.5 nm thick was then vapor deposited on this electron transport layer, finally, aluminum of 100 nm thick was vapor deposited, and a sealing was provided under nitrogen atmosphere.

The resulting organic EL element was measured in terms of EL properties by applying a voltage between the ITO as a positive electrode and the aluminum electrode as a negative electrode. The voltage, peak wavelength of emission and current efficiency under a current density of 5 A/m² are shown in Table 2.

TABLE 2

| | | | | Current |
|---|---|---|---|---|
| | Luminescent | Voltage | Peak Wavelength of | Efficiency |
| | Material | (V) | Emission (nm) | (cd/A) |
| Ex. 16 | Pt(dpt)(obp) | 6.3 | 511 | 56.4 |
| Ex. 17 | Pt(dpt)(OH) | 6.4 | 509 | 54.8 |
| Ex. 18 | Pt(dpt)(taz) | 6.2 | 504 | 58.2 |
| Ex. 19 | Pt(dpt)(sbtz) | 6.4 | 511 | 53.0 |
| Ex. 20 | Pt(diqt)(obp) | 6.5 | 624 | 15.7 |
| Ex. 21 | Pt(diqt)(OH) | 6.5 | 619 | 14.5 |
| Ex. 22 | Pt(diqt)(taz) | 6.3 | 614 | 16.3 |
| Ex. 23 | Pt(diqt)(sbtz) | 6.6 | 628 | 12.9 |
| Ex. 24 | Pt(dppr)(obp) | 7.3 | 477 | 17.3 |
| Ex. 25 | Pt(dppr)(OH) | 7.5 | 475 | 16.3 |
| Ex. 26 | Pt(dppr)(taz) | 7.2 | 469 | 17.2 |
| Ex. 27 | Pt(dppr)(sbtz) | 7.6 | 480 | 14.6 |
| Ex. 28 | Pt(dpt)(acph) | 6.5 | 509 | 30.5 |
| Ex. 29 | Pt(dpt)(oph) | 6.1 | 509 | 59.9 |
| Ex. 30 | Pt(dpt)(obtz) | 6.2 | 508 | 55.6 |
| Ex. 31 | Pt(dpt)(obp) | 8.1 | 510 | 32.5 |

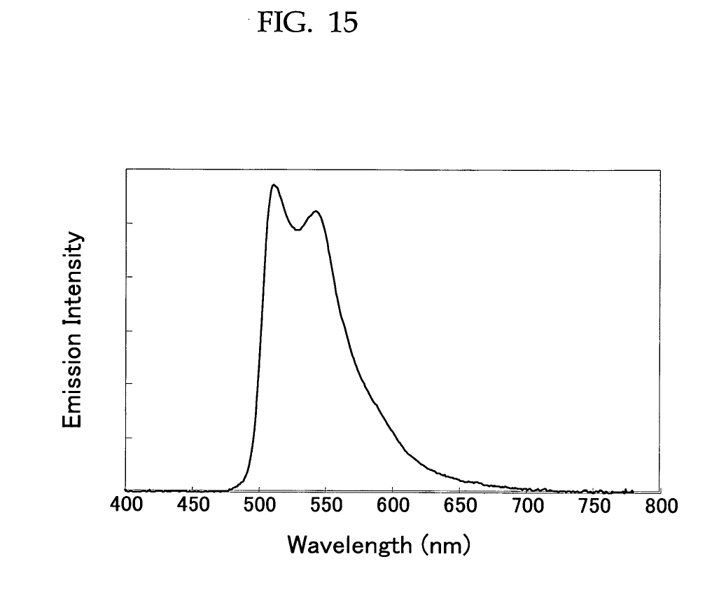

The results of Table 2 clearly demonstrate that the organic EL elements according to the present invention (Example 16 to 31) unexceptionally represent significantly higher EL efficiencies. FIG. 15 shows an EL spectrum of the element of Example 14.

### EXAMPLE 32

The organic EL element prepared in Example 16 was evaluated in terms of fluctuation of luminance brightness through a continuous driving at a current density of 50 A/m². The half life of luminance brightness was 70 hours from the initial luminance brightness of 2365 cd/m².

### EXAMPLE 33

The organic EL element prepared in Example 17 was evaluated in terms of fluctuation of luminance brightness through a continuous driving at a current density of 50 A/m². The half life of luminance brightness was 70 hours from the initial luminance brightness of 2365 cd/m².

### EXAMPLE 34

The organic EL element prepared in Example 18 was evaluated in terms of fluctuation of luminance brightness through a continuous driving at a current density of 50 A/m². The half life of luminance brightness was 75 hours from the initial luminance brightness of 2412 cd/m².

### EXAMPLE 35

The organic EL element prepared in Example 19 was evaluated in terms of fluctuation of luminance brightness through a continuous driving at a current density of 50 A/m². The half life of luminance brightness was 60 hours from the initial luminance brightness of 2055 cd/m².

### COMPARATIVE EXAMPLE 1

An organic EL element was prepared in the same manner as Example 16 except that the luminescent material of Pt(dpt)(obp) was changed into Pt(dpt)Cl. The resulting organic EL element was evaluated in terms of fluctuation of luminance brightness through a continuous driving at a current density of 50 A/m². The half life of luminance brightness was as short as 0.3 hour from the initial luminance brightness of 1877 cd/M².

### SYNTHESIS EXAMPLE 15

### Synthesis of Pt(1,3-dipyrazolylbenzene)(phenoxide) (hereinafter referred to as “Pt(dpzb)(oph)”)

Pyrazole (1.50 g, 22 mmol), 1,3-dichlorobenzene (1.47 g, 10 mmol), 2-dicyclohexylphosphino-2′,4′,6′-triisopropyl-1,1′-biphenyl (190 mg, 0.4 mmol), tris(dibenzylideneacetone)dipalladium (O) (92 mg, 0.02 nunol), KOH (1.68 g, 30 mmol), and water (10 ml) were charged into a round-bottom flask (50 ml), then the mixture was refluxed at 110° C. for 20 hours under nitrogen atmosphere while stirring. After cooling, the reaction product was subjected to an extraction using 500 ml of diethyether, dried over MgSO₄ powder, and diethyl ether was removed from the product using an evaporator. The resulting coarse product was recrystallized using dichloromethane to prepare 1.2 g of 1,3-(dipyrazolyl)benzene.

Thereafter, 1,3-(dipyrazolyl)benzene (1.05 g, 5 mmol), K₂PtCl₄ (2.49 g, 6 mmol) and acetic acid (150 ml) were charged into a round-bottom flask (500 ml), then the mixture was refluxed at 130° C. for 2 days while stirring under nitrogen atmosphere. A pale yellow crystal was deposited after cooling. The pale yellow crystal was then separated by filtration, washed sufficiently using methanol, water and diethylether, and dried under vacuum. The resulting coarse powder was recrystallized using dichloromethane to prepare 1.5 g of Pt(dpzb)Cl.

Then, Pt(dpzb)Cl (440 mg, 1 mmol) was added to acetone (150 ml) and the mixture was stirred. A solution of sodium phenoxide 3H₂O (272 mg, 1.6 mmol) and methanol (100 ml) was slowly added drop-wise and the mixture was stirred at room temperature for 10 minutes. Addition of a few drops of pure water promoted the reaction and initiated deposition of a pale yellow solid. The reactant was stirred for three hours while heating and allowed to cool then the deposited pale yellow solid was separated by filtration, washed sufficiently using pure water, methanol and diethylether in order, and dried under vacuum to obtain 400 mg of Pt(dpzb)(oph).

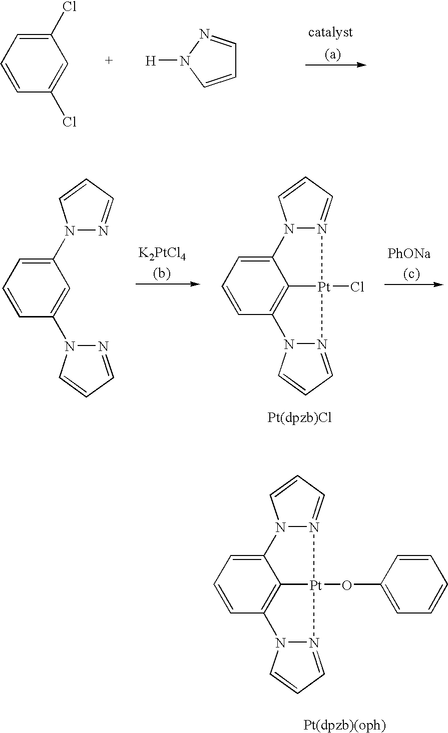

### SYNTHESIS EXAMPLE 16

### Synthesis of Pt(1-fluoro-3,5-dipyridylbenzene)(phenoxide) (hereinafter referred to as “Pt(fdpb)(oph)”)

Pt(fdpb)(oph) was synthesized in the same manner as Synthesis Example 1 except that 3,5-dibromotoluene was changed into 1,3-dibromo-5-fluorobenzene.

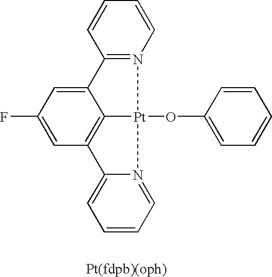

### SYNTHESIS EXAMPLE 17

### Synthesis of Pt(3,5-dipyridyltoluene)(diphenylphosphide) (hereinafter referred to as “Pt(dpt)(pdph)”)

Pt(dpt)(pdph) was synthesized in the same manner as Synthesis Example 3 except that 1,2,4-triazole sodium salt was changed into lithium diphenylphosphide.

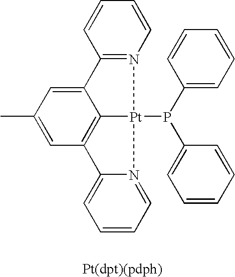

### SYNTHESIS EXAMPLE 18

### Synthesis of Pt(1,3-dibenzothiazolylbenzene)(phenoxide) (hereinafter referred to as “Pt(dbztb)(oph)”)

Into a round-bottom flask (300 ml), 2-bromobenzothiazole (4.71 g, 22 mmol), 1,3-phenylene bisboronic acid (1.66 g, 10 mmol), tetrakis(triphenylphosphine)palladium (O) (578 mg, 0.5 mmol), toluene (50 ml), ethanol (25 ml) and 2M Na₂CO₃ aqueous solution (50 ml) were added and the mixture was refluxed at 110° C. for 5 hours while stirring under nitrogen atmosphere. After cooling, the reactant liquid was poured into a large amount of water, the reactant product was extracted into toluene (500 ml) and dried over MgSO₄ powder, from which then toluene was removed by an evaporator. The resulting coarse product was subjected to recrystallization thereby to produce 1,3-dibenzothiazolylbenzene (2.8 g). Pt(dbztb)(oph) was produced in the same manner as Synthesis Example 15 using the resulting 1,3-dibenzothiazolylbenzene.

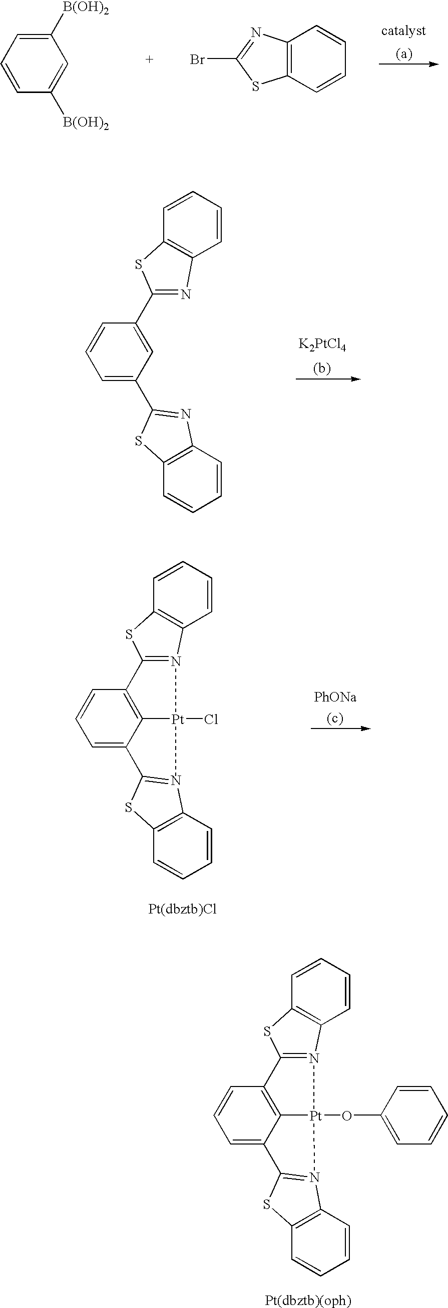

### COMPARATIVE SYNTHETIC EXAMPLE 1

### Synthesis of Pt(6-phenyl-2,2′-bipyridine)(phenylacetylide) (hereinafter referred to as “Pt(phbp)(acph)”)

Pt(phbp)(acph) was synthesized in accordance with the method described in JP-A No. 2002-363552.

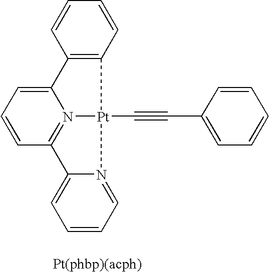

### EXAMPLES 36 TO 39 AND COMPARATIVE EXAMPLE 2

Thin films of luminescent materials were formed in the same manner as Example 1 except that the organometallic complex Pt(dpt)(obp) was changed into the organometallic complexes of Table 3; and their phosphorescent quantum yields were measured with respect to the resulting thin films of luminescent solids under the same conditions as Example 1. The results are shown in Table 3.

TABLE 3

| | Light Emitting | PL Quantum Yield |
|---|---|---|
| | Material | (%) |
| Ex. 36 | Pt(dpzb)(oph) | 90 |
| Ex. 37 | Pt(fdpb)(oph) | 92 |
| Ex. 38 | Pt(dpt)(pdph) | 95 |
| Ex. 39 | Pt(dbztb)(oph) | 92 |
| Com. Ex. 2 | Pt(phbp)(acph) | 8 |

### EXAMPLES 40 TO 43 AND COMPARATIVE EXAMPLE 3

Organic EL elements were prepared in the same manner as Example 16 except that the luminescent material was changed into those shown in Table 4. The resulting elements were measured in terms of EL properties by applying a voltage between the ITO as a positive electrode and the aluminum electrode as a negative electrode. The voltage, peak wavelength of emission and current efficiency under a current density of 5 A/m² are shown in Table 4.

TABLE 4

| | | | | Current |
|---|---|---|---|---|
| | Luminescent | Voltage | Peak Wavelength | Efficiency |
| | Material | (V) | of Emission (nm) | (cd/A) |
| Ex. 40 | Pt(dpzb)(oph) | 6.4 | 430 | 12.8 |
| Ex. 41 | Pt(fdpb)(oph) | 6.5 | 475 | 18.1 |
| Ex. 42 | Pt(dpt)(pdph) | 6.3 | 508 | 55.5 |
| Ex. 43 | Pt(dbztb)(oph) | 6.3 | 541 | 45.2 |
| Com. Ex. 3 | Pt(phbp)(acph) | 6.5 | 565 | 4.5 |

### INDUSTRIAL APPLICABILITY

The present invention can solve the problems in the art, that is, the present invention provides organometallic complexes capable of emitting phosphorescence and appropriately utilized for organic EL elements, luminescent materials in lighting systems, color conversion materials etc; organic EL elements, which containing the organometallic complex and/or the luminescent solid, and can exhibit longer durability, higher emitting efficiency, superior thermal/electrical stability, significantly longer operating life; organic EL displays, which containing the organic EL element, and can exhibit higher performance and longer durability, represent a constant average driving current regardless of the luminous pixel, be appropriately utilized for full-color displays with excellent color balance without changing the emitting area, and represent longer operating life.

The organometallic complexes or luminescent materials according to the present invention are phosphorescent, and may be appropriately utilized as luminescent materials, color transfer materials etc. in organic EL elements or lighting systems.

The organic EL elements according to the present invention include the organometallic complexes, thus can exhibit longer durability, higher emitting efficiency, superior thermal/electrical stability, excellent color transfer efficiency and significantly longer operating life; as such, the organic EL elements may be suitably used in various applications such as televisions, cellular phones, computers, display devices in vehicles, field display devices, home apparatuses, industrial apparatuses, household electric appliances, traffic display devices, clock display devices, calendar display units, luminescent screens, audio equipment, lighting systems and also organic EL displays described below in particular.

The organic EL displays according to the present invention includes the organic EL elements thus can exhibit higher performance and longer durability, and may be suitably used in various fields such as televisions, cellular phones, computers, display devices for vehicle mounting, field display devices, home apparatuses, industrial apparatuses, household electric appliances, traffic display devices, clock display devices, calendar display units, luminescent screens and audio equipment.

1. An organometallic complex, expressed by the general formula (1) below: 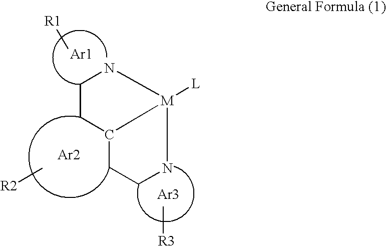
in the general formula (1), M represents a metal; Ar1, Ar2 and Ar3 each represents a ring structure; Ar1 and Ar3 are identical; R1, R2 and R3 may be identical or different each other, each represents a hydrogen atom or a substituent; R1, R2 and R3 may be each plural hydrogen atoms or substituents which may form a ring structure from adjacent ones; and L represents a monodentate ligand that bonds with the metal atom through a atom selected from the group consisting of C, N, O P and S atoms.

2. The organometallic complex according to claim 1, wherein the three atoms of the two nitrogen atoms and the one carbon atom in the tridentate ligand are each a part of ring structures which are different each other.

3. The organometallic complex according to claim 1,
wherein the first nitrogen-adjacent atom bonds to the first carbon-adjoining atom, in which the first nitrogen-adjacent atom indicates the atom adjacent to the first nitrogen atom in the ring structure, and the first carbon-adjoining atom indicates the first atom adjoining to the carbon atom in the ring structure; and
the second nitrogen-adjacent atom bonds to the second carbon-adjoining atom, in which the second nitrogen-adjacent atom indicates the atom adjacent to the second nitrogen atom in the ring structure, and the second carbon-adjoining atom indicates the second atom adjoining to the carbon atom in the ring structure.

4. The organometallic complex according to claim 3, wherein the first carbon-adjoining atom and the second carbon-adjoining are each a carbon atom.

5. (canceled)

6. The organometallic complex according to claim 1, wherein Ar1, Ar2 and Ar3 are each selected from five-membered ring groups, six-membered ring group and condensed ring groups thereof.

7. The organometallic complex according to claim 1, wherein Ar2 is at least one of benzene ring structures, pyridine ring structures, pyrimidine ring structures and pyrene ring structures.

8. The organometallic complex according to claim 1, wherein at least one of Ar1 and Ar3 is one of monocyclic heteroaromatic groups and polycyclic heteroaromatic groups.

9. (canceled)

10. The organometallic complex according to claim 1, wherein the metal atom is at least one selected from the group consisting of Fe, Co, Ni, Ru, Rh, Pd, W, Re, Os, Ir and Pt.

11. The organometallic complex according to claim 1, wherein the organometallic complex is electrically neutral.

12. The organometallic complex according to claim 1, wherein the organometallic complex sublimes under vacuum.

13. The organometallic complex according to claim 1, wherein the organometallic complex is used for one of organic EL elements and lighting systems.

14. A luminescent solid, comprising the organometallic complex according to claim 1.

15. An organic EL element, comprising an organic thin film layer between a positive electrode and a negative electrode,
wherein the organic thin film layer comprises an organometallic complex, expressed by the general formula (1) below: 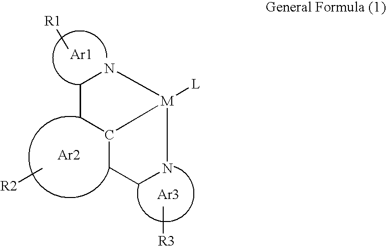
in the general formula (1), M represents a metal; Ar1, Ar2 and Ar3 each represents a ring structure; Ar1 and Ar3 are identical; R1, R2 and R3 may be identical or different each other, each represents a hydrogen atom or a substituent; R1, R2 and R3 may be each plural hydrogen atoms or substituents which may form a ring structure from adjacent ones; and L represents a monodentate ligand that bonds with the metal atom through a atom selected from the group consisting of C, N, O, P and S atoms.

16. The organic EL element according to claim 15, wherein the organic thin film layer comprises a light emitting layer interposed between a positive hole transport layer and a electron transport layer, and the light emitting layer comprises an organometallic complex as the luminescent material.

17. The organic EL element according to claim 16, wherein the light emitting layer is formed by making the organometallic complex by itself into a film.

18. The organic EL element according to claim 16, wherein the light emitting layer comprises a carbazole derivative expressed by the structural formula (2) below: 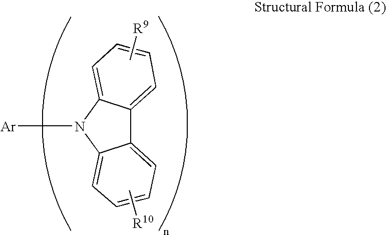
in the structural formula (2), Ar represents a divalent or a trivalent group containing an aromatic ring, or a divalent or a trivalent group containing a heterocyclic aromatic ring; R⁹ and R¹⁰ represent each independently a hydrogen atom, halogen atom, alkyl group, aralkyl group, alkenyl group, aryl group, cyano group, amino group, acyl group, alkoxycarbonyl group, carboxyl group, alkoxy group, alkylsulfonyl group, hydroxyl group, amide group, aryloxy group, aromatic hydrocarbon or aromatic heterocyclic group, which may be further substituted by a substituent group; n represents an integer of 2 or 3.

19. The organic EL element according to claim 16, wherein the electron transport material in the electron transport layer is 2,9-dimethyl-4,7-diphenyl-1,10-phenanthroline (BCP) expressed by the structural formula (68) below.

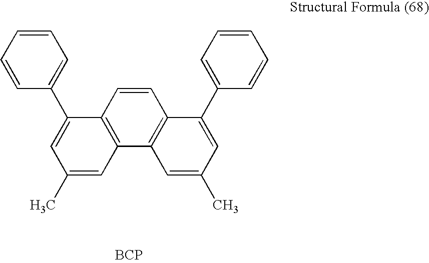

20. The organic EL element according to claim 15, utilized for emitting a red light.

21. An organic EL display, comprising an organic EL element according to claim 15.

22. The organic EL display according to claim 21, wherein the organic EL display is used for one of passive matrix panels and active matrix panels.
